(19) United States
(12) Patent Application Publication
(10) Pub. No.: US 2024/0316149 A1
(43) Pub. Date: Sep. 26, 2024

(54) HEMOGLOBIN-BASED OXYGEN CARRIERS AND METHODS OF MAKING AND USING THEREOF

(71) Applicants: Ohio State Innovation Foundation, Columbus, OH (US); THE REGENTS OF THE UNIVERSITY OF CALIFORNIA, Oakland, CA (US)

(72) Inventors: Andre PALMER, Westerville, OH (US); Pedro J. CABRALES AREVALO, La Mesa, CA (US); Xiangming GU, Columbus, OH (US); Chintan SAVLA, Columbus, OH (US)

(21) Appl. No.: 18/574,173

(22) PCT Filed: Jun. 24, 2022
(86) PCT No.: PCT/US22/34984

§ 371 Date: Dec. 26, 2023

Related U.S. Application Data
(60) Provisional application No. 63/214,593, filed on Jun. 24, 2021.

(51) Int. Cl. A61K 38/17; A01N 1/02; A61K 47/54; A61K 47/60; A61K 47/61; A61K 47/64

(52) U.S. Cl. CPC A61K 38/1722; A01N 1/0226; A61K 47/54; A61K 47/60; A61K 47/61; A61K 47/64; A61K 47/6445

(57) ABSTRACT

Disclosed are hemoglobin-based oxygen carriers (HBOCs) that comprise a polymer-functionalized polymerized hemoglobin, such as a polyalkylene oxide (PAO)-functionalized polymerized hemoglobin, as well as compositions comprising these HBOCs (e.g., dissolved or dispersed in an aqueous carrier) and methods of making and using thereof.

### CROSS REFERENCE TO RELATED APPLICATIONS

This application claims the benefit of priority to U.S. Provisional Application No. 63/214,593 filed Jun. 24, 2021, which is incorporated by reference herein in its entirety.

### STATEMENT REGARDING FEDERALLY SPONSORED RESEARCH OR DEVELOPMENT

This invention was made with government support under grant/contract numbers R01 HL156526, R01 HL138116, R01 HL126945, R01 EB021926, and R01 HL159862 awarded by the National Institutes of Health. The government has certain rights in the invention.

### BACKGROUND

Currently, there is a shortage in the number of red blood cell (RBC) units available for blood transfusions. Donated human red blood cells (currently the only source for these transfusions) do not adequately meet current demands and are unlikely to meet future demands. For example, in the United States, 14 million red blood cell units are made available for transfusions each year, and an annual deficit of 1 million units still exists. Internationally, the deficit is around 200 million units annually. In the future, these deficits may become even more severe, as the current deficit projections do not take into account the more acute need for blood in cases of mass civilian casualties, such as natural disasters, terrorist attacks and wars.

The shortage in red blood cell units and the lack of suitable substitutes results in many preventable deaths. For example, more than 530,000 women die each year during pregnancy or childbirth, with hemorrhage being the leading cause of death (accounting for up to 44% of maternal deaths in some areas of sub-Saharan Africa). Many of these deaths could be prevented with the proper supply of red blood cell units or with a proper substitute for red blood cell transfusions.

While the current deficits are in part due to a shortage in the number of blood donors, other factors also contribute to the current crisis. Several of these factors relate to the pool of available donated human red blood cells being limited by existing obstacles to safe transfusion. For example, red blood cell units may carry infectious diseases and many multi-level proactive interventional programs of stringent red blood cell donor screening and expensive nucleic acid testing procedures have been implemented to protect recipients. With each new emerging disease, new diagnostic tests are performed, further limiting the available donor pool and further increasing costs. In addition, donated human red blood cells contain proteins and cytokines that may cause reactions in up to 2% of transfusions with symptoms ranging in severity from mild allergic reactions to severe shock and even death. Also, red blood cell transfusions may lead to various metabolic conditions (e.g., hyperkalemia, hypocalcemia and alkalosis), and multiple red blood cell transfusions may exert an immunosuppressive effect on the recipient, increasing risk of hospital-acquired infections. These potential complexities further increase costs associated with donor red blood cell transfusions.

In addition to the above-mentioned obstacles to safe transfusion, costs are also increased due to requirements for cross-matching donor and recipient red blood cell units before transfusion, as well as the short storage life (typically 15 days) and expensive storage requirements (e.g., must be kept at 2-3° C., special storage solutions are required to extend red blood cell life to 42 days, etc.) of red blood cell units. While there have been many recent improvements in technology, the collection and storage of donated red blood cells remains a difficult and expensive task. There is a need for a less expensive and more effective alternative to donated human red blood cell transfusions.

### SUMMARY

Described herein are hemoglobin-based oxygen carriers (HBOCs) that comprise a polymer-functionalized polymerized hemoglobin, as well as compositions comprising the same (e.g., dissolved or dispersed in an aqueous carrier).

In some embodiments, the polymer-functionalized polymerized hemoglobin can have a weight average molecular weight of from 500 kDa to 2,000 kDa, as determined by size exclusion (SEC) HPLC. The polymer-functionalized polymerized hemoglobin can be substantially free of low-molecular weight hemoglobin species having a molecular weight of less than 100 kDa (e.g, substantially free of low-molecular weight hemoglobin species having a molecular weight of less than 250 kDa, or substantially free of low-molecular weight hemoglobin species having a molecular weight of less than 500 kDa).

In some embodiments, the polymer-functionalized polymerized hemoglobin exhibits an average hydrodynamic diameter of from 13 nm to 100 nm (e.g., from 13 nm to 50 nm, or from 13 nm to 30 nm), as measured by dynamic light scattering.

In some embodiments, the polymer-functionalized polymerized hemoglobin exhibits a zeta potential of from −20 mV to less than 0 mV (e.g., from −10 mV to less than 0 mV).

The polymerized hemoglobin can comprise hemoglobin crosslinked with a multifunctional crosslinking agent, such as a dialdehyde (e.g., glutaraldehyde). For example, in some embodiments, the polymerized hemoglobin can be formed by a process that comprises crosslinking hemoglobin with a dialdehyde, such as glutaraldehyde, at a molar ratio of dialdehyde:hemoglobin of from 20:1 to 35:1. In some embodiments, the hemoglobin can be substantially in the T-state (tense quaternary state) during the crosslinking. In other embodiments, the hemoglobin can be substantially in the R-state (relaxed quaternary state) during the crosslinking.

In some embodiments, the polymerized hemoglobin can further comprise one or more antioxidant proteins co-polymerized with the hemoglobin. The one or more antioxidant proteins can modulate the autooxidation rate of the hemoglobin present in the HBOC. In some examples, the one or more antioxidant proteins can comprise antioxidant proteins present in red blood cells, such as a peroxiredoxin (e.g., peroxiredoxin-1, -2, and/or -6), a superoxide dismutase, a catalase, or a combination thereof.

The polymer-functionalized polymerized hemoglobin can comprise a polymer or oligomer covalently conjugated to the polymerized hemoglobin. Any suitable polymer or oligomer can be used. For example, in some embodiments, the polymer or oligomer can comprise a polyalkylene oxide, such as a polyethylene glycol (PEG), a zwitterionic polymer, such as a polycarboxybetaine (PCB) or a polysulfobetaine (PSB), a carbohydrate such as a dextran, or any combination thereof.

In certain embodiments, the polymer-functionalized polymerized hemoglobin comprises a polyalkylene oxide (PAO)-functionalized polymerized hemoglobin. The PAO-functionalized polymerized hemoglobin can comprise a polyalkene oxide (e.g., polyethylene glycol (PEG)) covalently conjugated to the polymerized hemoglobin. For example, is some embodiments, the PAO is polyethylene glycol (PEG) according to the formula of H(OCH₂CH₂)ₙOH, where n is greater than or equal to 4 (e.g., from 10 to 250, or from 75 to 125).

In some embodiments, the PAO-functionalized polymerized hemoglobin comprises polymerized hemoglobin conjugated with malemidyl-activated polyethylene glycol (Mal-PEG), as defined by Formula I below

PolyHb-(S—Y—R—CH₂—CH₂—[O—CH₂—CH₂]ₙ—O—CH₃)ₘ Formula I

wherein PolyHb represents polymerized hemoglobin; S represents a surface thiol group; Y represents a covalent bond between the polymerized hemoglobin and PEG; R is a linker; n is an integer of from 10 to 250, such as from 75 to 125; and m is an integer of greater than 2.

In some embodiments, the polymer-functionalized polymerized hemoglobin was polymerized in the T-state (tense quaternary state). In certain embodiments, the polymer-functionalized polymerized hemoglobin exhibits a P₅₀ of from 15 mm Hg to 40 mm Hg, a k_{off,O2} of from 10 s⁻¹ to 40 s⁻¹, or a combination thereof.

In other embodiments, the polymer-functionalized polymerized hemoglobin was polymerized in the R-state (relaxed quaternary state). In certain embodiments, the polymer-functionalized polymerized hemoglobin exhibits a P₅₀ of 1.0±0.5 mm Hg, a k_{off,O2} of from 7 s⁻¹ to 20 s⁻¹, or a combination thereof.

Also provided are methods of producing a hemoglobin-based oxygen carrier (HBOC). These methods can comprise. (i) contacting hemoglobin with a multifunctional cross-linking agent to form a solution comprising polymerized hemoglobin; (ii) filtering the solution comprising polymerized hemoglobin by ultrafiltration (e.g., tangential flow filtration) against a first filtration membrane having a pore size that separates the polymerized hemoglobin from low-molecular weight hemoglobin species, reactants, and reaction byproducts, thereby forming a retentate fraction comprising the polymerized hemoglobin and a permeate fraction comprising impurities; (iii) covalently conjugating one or more polymers to the polymerized hemoglobin to form a solution comprising a polymer-functionalized polymerized hemoglobin; and (iv) filtering the solution comprising the polymer-functionalized polymerized hemoglobin by ultrafiltration (e.g., tangential flow filtration) against a second filtration membrane having a pore size that separates the polymer-functionalized polymerized hemoglobin from low-molecular weight impurities, reactants, and reaction byproducts, thereby forming a retentate fraction comprising the polymer-functionalized polymerized hemoglobin and a permeate fraction comprising impurities.

In some embodiments, step (i) can comprise deoxygenating the hemoglobin such that substantially all of the hemoglobin is in the T-state (tense quaternary state) prior to contacting the hemoglobin with the multifunctional cross-linking agent. In other embodiments, step (i) can comprise oxygenating the hemoglobin such that substantially all of the hemoglobin is in the R-state (relaxed quaternary state) prior to contacting the hemoglobin with the multifunctional cross-linking agent.

In some embodiments, the multifunctional cross-linking agent can comprise a dialdehyde, such as glutaraldehyde. The multifunctional cross-linking agent and the hemoglobin are present at a molar ratio of dialdehyde:hemoglobin of from 20:1 to 35:1.

In some examples, the hemoglobin utilized in step (i) can further comprise one or more antioxidant proteins which also react with the multifunctional cross-linking agent, thereby becoming co-polymerized with the hemoglobin. The one or more antioxidant proteins can comprise antioxidant proteins present in red blood cells, such as a peroxiredoxin (e.g., peroxiredoxin-1, -2, and/or -6), a superoxide dismutase, a catalase, or a combination thereof. In some examples, step (i) can be performed using a clarified red blood cell lysate which includes a mixture of hemoglobin, antioxidant proteins, and optionally one or more additional proteins found in red blood cells.

The first filtration membrane can be rated for removing solutes having a molecular weight less than the molecular weight of the polymerized hemoglobin. In some examples, the first filtration membrane is rated for removing solutes having a molecular weight of from 1 to 500 kDa, from 1 to 250 kDa, from 1 to 100 kDa, from 1 to 50 kDa, or from 1 to 10 kDa.

In some embodiments, step (ii) can further comprise filtering the solution comprising polymerized hemoglobin by ultrafiltration (e.g., tangential flow filtration) against a third filtration membrane having a pore size that separates the polymerized hemoglobin from high-molecular weight hemoglobin species, reactants, and reaction byproducts, thereby forming a permeate fraction comprising the polymerized hemoglobin and a retentate fraction comprising impurities.

In some examples, the third filtration membrane can be rated for retaining solutes having a molecular weight greater than the molecular weight of the polymerized hemoglobin. For example, the third filtration membrane can be rated for retaining solutes having a molecular weight of at least 500 kDa, at least 750 kDa, at least 1000 kDa, or more. In certain embodiments, the third filtration membrane can have a pore size of at least about 0.1 μm, such as a pore size of about 0.2 μm.

In some embodiments, following step (ii), substantially all of the polymerized hemoglobin has a molecular weight of at least 100 kDa, such as at least 250 kDa or at least 500 kDa. For example, in certain embodiments, following step (ii), substantially all of the polymerized hemoglobin has a molecular weight of from 100 kDa to 10,000 kDa, such as from 100 kDa to 500 kDa, from 100 kDa to 750 kDa, from 100 kDa to 1,000 kDa, from 100 kDa to 5,000 kDa, from 250 kDa to 500 kDa, from 250 kDa to 750 kDa, from 250 kDa to 1,000 kDa, from 250 kDa to 5,000 kDa, 250 kDa to 10,000 kDa, from 500 kDa to 750 kDa, from 500 kDa to 1,000 kDa, from 500 kDa to 5,000 kDa, 500 kDa to 10,000 kDa, from 750 kDa to 1,000 kDa, or from 750 kDa to 5,000 kDa, or from 750 kDa to 10,000 kDa.

Step (iii) can comprise covalently conjugating one or more polyalkylene oxides polymers, such as one or more polyethylene glycol (PEG) polymers, to the polymerized hemoglobin to form a solution comprising a polyalkylene oxide (PAO)-functionalized polymerized hemoglobin, such as a polyethylene glycol (PEG)-functionalized polymerized hemoglobin. For example, in some embodiments, step (iii) can comprise contacting the polymerized hemoglobin with a thiolating reagent (e.g., 2-iminothiolane, Traut's reagent) and a malemidyl-activated PAO, such as a malemidyl-activated polyethylene glycol (Mal-PEG).

The second filtration membrane can be rated for removing solutes having a molecular weight less than the molecular weight of the polymer-functionalized polymerized hemoglobin. In some examples, the second filtration membrane is rated for removing solutes having a molecular weight of from 1 to 750 kDa, from 1 to 500 kDa, from 1 to 250 kDa, or from 1 to 100 kDa.

The HBOCs described herein (as well as compositions comprising these HBOCs) can be administered to subjects in need thereof, for example, as a blood substitute. For example, in some embodiments, the HBOCs described herein (as well as compositions comprising these HBOCs) can be administered to subjects in need thereof to treat a loss of blood due to injury, hemolytic anemia, equine infectious anemia, feline infectious anemia, a bacterial infection, Factor IV fragmentation, hypersplenation, splenomegaly, hemorrhagic syndrome, hypoplastic anemia, aplastic anemia, idiopathic immune hemadytic conditions, iron deficiency, isoimmune hemdytic anemia, microangiopathic hemolytic anemia, parasitism, or any combination thereof.

### DESCRIPTION OF DRAWINGS

FIG. 1A schematically illustrates a multistage hollow fiber (HF) filtration system used to purify Hb.

FIG. 1B schematically illustrates the systems used for the synthesis and fractionation of T- and R-state PolybHb starting from bHb

FIG. 2 schematically illustrates the systems and methds for the synthesis and purification of PEG-PolybHb starting from purified LMW and HMW T- and R-state PolybHb. The reaction mixture was allowed to react for 16 hours or overnight at 4° C. TFF was then used to eliminate unreacted reagents and to perform buffer exchange into PBS.

FIGS. 3A-3D show the size and MW analysis of T-state and R-state PolybHbs and PEG-PolybHbs. FIGS. 3A and 3B show SEC-HPLC chromatograms for bHb, T- and R-state PolybHbs, and corresponding PEGylated PolybHbs. A left shift in elution time and increase in the apparent MW was observed for all species after PEGylation. FIGS. 3C and 3D show DLS curves for all species. An increase in hydrodynamic diameter was observed for all species after PEGylation. Unmodified bHb was used as a control.

FIGS. 4A-4D illustrate the O₂ equilibria and O₂ offloading kinetics of T-state and R-state PolybHbs and PEG-PolybHbs. FIGS. 4A and 4B show the O₂ equilibrium curves for bHb, LMW and HMW T-state and R-state PolybHbs, and LMW and HMW T-state and R-state PEG-PolybHbs, respectively. Lines represent the mean from all batches. FIGS. 4C and 4D show the normalized deoxygenation kinetics of bHb, LMW and HMW T-state and R-state PolybHbs, and LMW and HMW T-state and R-state PEG-PolybHbs, respectively. The absorbance was monitored at 437.5 nm and normalized against the maximum value.

FIGS. 5A-5D illustrate PolybHb and PEG-PolybHb Hp binding and auto-oxidation kinetics. FIG. 5A illustrates pseudo first order Hp-PolybHb/PEG-PolybHb binding kinetics. The normalized fluorescence changes were fit to a monoexponential equation. FIG. 5B shows second order Hp binding kinetics. The second order Hp binding rate constants were obtained by performing a linear fit of the pseudo first order Hp binding rate constants as a function of PolybHb/PEG-PolybHb concentration. FIGS. 5C and 5D show the auto-oxidation kinetics of bHb, R-state (FIG. 5C) and T-state (FIG. 5D) PolybHb/PEG-PolybHb. Auto-oxidation of 0.775 mM PolybHb/PEG-PolybHb was measured via UV-visible spectrometry in 50 mM PB pH 7.4 at 37° C. for 24 hr.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is processed (e.g., continually processed) downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

The terms “isolating,” “purifying,” and “separating,” as used interchangeably herein, refer to increasing the degree of purity of a polypeptide or protein of interest or a target protein from a composition or sample comprising the polypeptide and one or more impurities (e.g., additional proteins or polypeptides).

### Hemoglobin-Based Oxygen Carriers (HBOCs) and Compositions

Described herein are hemoglobin-based oxygen carriers (HBOCs) that comprise a polymer-functionalized polymerized hemoglobin, as well as compositions comprising the same (e.g., dissolved or dispersed in an aqueous carrier).

As mentioned above, the collection and storage of donated red blood cells for transfusion is a difficult and expensive task. Artificial blood substitutes are a potential alternative to donor blood and provide several advantages over human donor blood. For example, artificial blood substitutes may be: designed to be free of human red blood cell antigens (i.e., can be administered to individuals possessing any blood group type); readily mass-produced with guaranteed sterility (eliminating the possibility of infectious transmittal or the need for infectious blood screening); designed to have longer storage lifetimes and require less stringent storage conditions than donor blood; and produced at lower costs (e.g., by avoiding the screening and storage costs currently associated with human donor blood units).

The various embodiments provide compositions and methods for developing oxygen carriers which may be used, for example, as artificial blood substitutes that may include hemoglobin and/or hemoglobin derivatives.

In order to be an adequate replacement for donor blood, an ideal artificial blood substitute should replicate blood's ability to transport oxygen to tissues. For example, an ideal artificial blood substitute should be an oxygen therapeutic. An ideal synthetic oxygen therapeutic (i.e., oxygen-carrying artificial blood substitutes) should have normal physiological oxygen-binding properties, be uniform and small size so as to both afford long circulation lifetimes and safe clearance from body, have human bloodlike viscosity and oncotic pressure characteristics so as to preserve shear forces in the microcirculation and enable plasma expansion in the resuscitation of patients, have tunable oxygen release parameters for tissues experiencing normal or low oxygenation, and be free of infectious disease risks associated with intravenous administration.

Various embodiments provide an oxygen-carrying artificial blood substitute that has normal physiological oxygen-binding properties, is uniform and small size, has human bloodlike viscosity and oncotic pressure characteristics, has tunable oxygen release parameters, and is resistant to infectious diseases. The HOBCs and compositions described herein can provide for safe and effective oxygen delivery.

While existing hemoglobin-based oxygen therapeutics have numerous advantageous over the perfluorocarbons-based oxygen therapeutics, initial studies involving the infusion of cell-free hemoglobin into animals, showed that free hemoglobin results in significant vasoconstriction and kidney damage. Consequently, hemoglobin-based oxygen carriers (HBOCs) often require stabilizing the hemoglobin molecule in order to eliminate adverse physiological effects while maintaining the physiological oxygen-transporting ability of native cell-free hemoglobin Existing HBOCs can induce vasoconstriction when transfused into animals due to nitric oxide (NO) sequestration and/or an over-oxygenation auto-regulatory response. Moreover, existing HBOCs generally demonstrate limited circulatory half-lives (usually less than 12 hours) and are only suitable for short-term applications. The various embodiments provide an oxygen carrier that maintains the physiological oxygen-transporting abilities of native cell-free hemoglobin while avoiding the adverse physiological effects associated with hemoglobin and existing HBOCs.

HBOCs comprise a polymer-functionalized polymerized hemoglobin. Hemoglobin (Hb) is the oxygen-carrying component of blood that circulates through the bloodstream inside small enucleate cells known as erythrocytes or red blood cells. It is a protein comprised of four associated polypeptide chains that bear prosthetic groups known as hemes. The structure of hemoglobin is well known and described in Bunn & Forget, eds., Hemoglobin: Molecular, Genetic and Clinical Aspects (W. B. Saunders Co., Philadelphia, Pa.: 1986) and Fermi & Perutz “Hemoglobin and Myoglobin,” in Phillips and Richards, Atlas of Molecular Structures in Biology (Clarendon Press: 1981). As blood circulates through the lungs, the oxygen present in the alveolar capillaries diffuses through the alveolar membrane and acts to convert virtually all of the hemoglobin within the red cells to a reversible molecular complex known as oxyhemoglobin. During this oxygenation process, the red blood cells become cherry red in color. Because the association of the oxygen and hemoglobin molecules within the red cells is reversible, the oxygen molecules are gradually released from the hemoglobin molecules (or from the red blood cells) when blood reaches the tissue capillaries. Eventually, the oxygen molecules diffuse into the tissues and is consumed by metabolism. As the oxyhemoglobin releases its bound oxygen, the red cells become purple in color.

As used herein, the term “hemoglobin” refers to the iron-containing oxygen-transport metalloprotein in the red blood cells of all vertebrates. Hemoglobin can be obtained from a variety of mammalian sources, such as, for example, human, or bovine (genus bos), or bison (genus bison), or ovine (genus ovis), or porcine (genus sus) sources, or other vertebrates or as transgenically-produced hemoglobin. Alternatively, the hemoglobin for use in the methods and compositions described herein can be synthetically produced by a bacterial cell, or more preferably, by a yeast cell, mammalian cell, or insect cell expression system (Hoffman, S. J. et al., U.S. Pat. No. 5,028,588 and Hoffman, et al., WO 90/13645, both herein incorporated by reference). Alternatively, hemoglobin can be obtained from transgenic animals; such animals can be engineered to express non-endogenous hemoglobin (Logan, J. S. et al. PCT Application No. PCT/US92/05000; Townes, T. M. et al., PCT Application No. PCT/US/09624, both herein incorporated by reference in their entirety).

Hemoglobin can also encompass genetically modified and/or recombinantly produced hemoglobin as well as chemically treated or surface decorated hemoglobins either in their dimeric, or tetrameric or variously polymerized forms. Expression of various recombinant hemoglobins has been achieved. Such expression methods include individual globin expression as described, for example, in U.S. Pat. No. 5,028,588, and di-alpha globin expression created by joining two alpha globins with a glycine linker through genetic fusion coupled with expression of a single beta globin gene to produce a pseudotetrameric hemoglobin molecule as described in WO 90/13645 and Looker et al., Nature 356:258 260 (1992). Other modified recombinant hemoglobins are disclosed in PCT Publication WO 96/40920. Similar to other heterologous proteins expressed in E. coli, recombinant hemoglobins have N-terminal methionines, which in some recombinant hemoglobins replace the native N-terminal valines.

In some embodiments, the hemoglobin is from a mammalian, invertebrate, or recombinant source. In certain embodiments, the hemoglobin is from a mammalian source. For example, the hemoglobin can comprise bovine hemoglobin, procine hemoglobin, or human hemoglobin. In certain embodiments, the hemoglobin can comprise recombinantly produced hemoglobin. In other embodiments, the hemoglobin can comprise chemically or genetically modified hemoglobin that, for example, prevent dissociation of the hemoglobin molecule or modify the oxygen-binding affinity.

In some embodiments, the hemoglobin can be purified using ultrafiltration (e.g., tangential flow filtration) prior to polymerization. For example, in some cases, the hemoglobin can be purified using a multistage tangential flow filtration process, such as that described in Palmer, A. F.; Sun, G.; Harris, D. R. Tangential Flow Filtration of Hemoglobin. Biotechnol. Prog. 2009, 25 (1), 189-199.

The hemoglobin present in the HBOCs described herein can be polymerized. The term “polymerized,” as used herein with respect to hemoglobin, encompasses both inter-molecular and intramolecular polyhemoglobin, with at least 50%, preferably greater than about 95%, of the polymerized hemoglobin of greater than tetrameric form. The polymerized hemoglobin can be prepared by polymerizing or cross-linking hemoglobin with a multifunctional cross-linking agent. Preferably, the polymerized hemoglobin is substantially soluble in aqueous fluids having a pH of 6 to 9 and in physiological fluids. Suitable examples of cross-linking agents are disclosed in U.S. Pat. No. 4,001,200, the entire teachings of which are incorporated herein by reference.

Suitable specific examples of the cross-linking agents include compounds having an aldehyde or dialdehyde functionality, such as formaldehyde, paraformaldehyde, formaldehyde activated ureas such as 1,3-bis(hydroxymethyl)urea, N,N′-di(hydroxymethyl) imidazolidinone prepared from formaldehyde condensation with a urea; compounds bearing a functional isocyanate or isothiocyanate group, such as diphenyl-4,4′-diisothiocyanate-2,2′-disulfonic acid, toluene diisocyanate, toluene-2-isocyanate-4-isothiocyanate, 3-methoxydiphenylmethane-4,4′-diisocyanate, propylene diisocyanate, butylene diisocyanate, and hexamethylene diisocyanate; esters and thioesters activated by strained thiolactones; hydroxysuccinimide esters; halogenated carboxylic acid esters; and imidates. Other examples of the cross-linking agents include derivatives of carboxylic acids and carboxylic acid residues of hemoglobin activated in situ to give a reactive derivative of hemoglobin that will cross-link with the amines of another hemoglobin. Examples of the carboxylic acids include citric, malonic, adipic and succinic acids. Carboxylic acid activators include thionyl chloride, carbodiimides, N-ethyl-5-phenyl-isoxazolium-3′-sulphonate (Woodward's reagent K), N,N′-carbonyldiimidazole, N-t-butyl-5-methylisoxazolium perchlorate (Woodward's reagent L), 1-ethyl-3-dimethyl aminopropylcarbodiimde, and 1-cyclohexyl-3-(2-moφpholinoethyl) carbodiimide metho-p-toluene sulfonate. The cross-linking reagent can be a dialdehyde precursor that readily forms a bifunctional dialdehyde in the reaction medium. Suitable dialdehyde precursors include acrolein dimer or 3,4-dihydro-1,2-pyran-2-carboxaldehyde which undergoes ring cleavage in an aqueous environment to give alpha-hydroxy-adipaldehyde. Other precursors, which on hydrolysis yield a cross-linking reagent, include 2-ethoxy-3,4-dihydro-1,2-pyran which gives glutaraldehyde, 2-ethoxy-4-methyl-3,4-dihydro-1,2-pyran which yields 3-methyl glutaraldehyde, 2,5-diethoxy tetrahydrofuran which yields succinic dialdehyde and 1,1,3,3-tetraethoxypropane which yields malonic dialdehyde and formaldehyde from trioxane. Exemplary commercially available cross-linking reagents include divinyl sulfone, epichlorohydrin, butadiene diepoxide, ethylene glycol diglycidyl ether, glycerol diglycidyl ether, dimethyl suberimidate dihydrochloride, dimethyl malonimidate dihydrochloride, and dimethyl adipimidate dihydrochloride.

Specific examples of the cross-linking agents include glutaraldehyde, succindialdehyde, activated forms of polyoxyethylene and dextran, c\ddot{v}-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class.

In some embodiments, the polymerized hemoglobin can comprise hemoglobin polymerized by a dialdehyde. As used herein, the “hemoglobin polymerized by a dialdehyde” includes both hemoglobin polymerized by a dialdehyde and hemoglobin polymerized by a dialdehyde precursor that readily forms a bifunctional dialdehyde in the reaction medium. Suitable dialdehyde and dialdehyde precursors are as described above. In some embodiments, the polymerized hemoglobin can comprise hemoglobin polymerized by glutaraldehyde.

The polymerized hemoglobin can be in the tense or relaxed quaternary state, or in between these two quaternary states. In some embodiments, the hemoglobin can be polymerized in the T-state (tense quaternary state). In other embodiments, the hemoglobin can be polymerized in the R-state (relaxed quaternary state).

The hemoglobin present in the HBOCs described herein can be polymer-functionalized. Polymer-functionalized polymerized hemoglobin can comprise a polymer or oligomer covalently conjugated to the polymerized hemoglobin. Any suitable polymer or oligomer can be used. For example, in some embodiments, the polymer or oligomer can comprise a polyalkylene oxide, such as a polyethylene glycol (PEG), a zwitterionic polymer, such as a polycarboxybetaine (PCB) or a polysulfobetaine (PSB), a carbohydrate such as a dextran, or any combination thereof.

In certain embodiments, the hemoglobin present in the HBOCs described herein can be polyalkylene oxide (PAO)-functionalized. Polyalkylene oxide (PAO)-functionalized hemoglobin comprises hemoglobin or polymerized hemoglobin that has been surface-modified with one or more polyalkylene oxide (PAO) polymers. In this context, “surface-modification” can refer to the covalent attachment of chemical groups (and ultimately PAO polymer chains) to one or more exposed amino acid side chains on the hemoglobin molecule. Modification can increase the molecular size of the hemoglobin.

Examples of suitable polyalkylene oxides include, but are not limited to, polyethylene oxide ((CH₂CH₂O)ₙ), polypropylene oxide ((CH(CH₃)CH₂O)ₙ), polybutylene oxide ((CH(CH₂CH₃)CH₂O)ₙ), and copolymers thereof such as polyethylene/polypropylene oxide copolymers ((CH₂CH₂O)ₙ—(CH(CH₃)CH₂O)ₙ). Such copolymers can include random copolymers, alternating copolymers, and block copolymers. The number of PEGs to be added to the polymerized hemoglobin may vary, depending on the size of the PEG.

In certain embodiments, the PAO is polyethylene glycol (PEG). PEGs are polymers of the general chemical formula H(OCH₂CH₂)ₙOH, where n is generally greater than or equal to 4. PEG formulations are usually followed by a number that corresponds to their average molecular weight. For example, PEG-200 has an average molecular weight of 200 and may have a molecular weight range of 190-210. PEGs are commercially available in a number of different forms, and in many instances come preactivated and ready to conjugate to proteins.

In some embodiments, polymerization and/or surface modification can take place when the hemoglobin is in the oxygenated or “R” state. This can be accomplished by allowing the hemoglobin to equilibrate with the atmosphere (or, alternatively, active oxygenation can be carried out) prior to polymerization and/or conjugation. By performing the polymerization and/or conjugation to oxygenated hemoglobin, the oxygen affinity of the resultant hemoglobin can be enhanced.

In one embodiment, the HBOC is polymerized hemoglobin to which malemidyl-activated PEG (“Mal-PEG”) has been conjugated. Such HBOCs may be further referred to by the following formula:

Hb-(S—Y—R—CH₂—CH₂—[O—CH₂—CH₂]ₙ—O—CH₃)ₘ Formula I

where Hb refers to polymerized hemoglobin, S is a surface thiol group, Y is the succinimido covalent link between Hb and Mal-PEG, R is an alkyl, amide, carbamate or phenyl group (depending on the source of raw material and the method of chemical synthesis), [O—CH₂—CH₂]n are the oxyethylene units making up the backbone of the PEG polymer, where n defines the length of the polymer (e.g., MW=5000), and O—CH₃ is the terminal methoxy group.

In some embodiments, the polymer-functionalized polymerized hemoglobin can have a weight average molecular weight of at least 500 kDa (e.g., at least 600 kDa, at least 700 kDa, at least 750 kDa, at least 800 kDa, at least 900 kDa, at least 1000 kDa, at least 1250 kDa, at least 1500 kDa, or at least 1750 kDa), as determined by size exclusion (SEC) HPLC. In some embodiments, the polymer-functionalized polymerized hemoglobin can have a weight average molecular weight of 2000 kDa or less (e.g., 1750 kDa or less, 1500 kDa or less, 1250 kDa or less, 1000 kDa or less, 900 kDa or less, 800 kDa or less, 750 kDa or less, 700 kDa or less, or 600 kDa or less), as determined by size exclusion (SEC) HPLC.

The polymer-functionalized polymerized hemoglobin can have a weight average molecular weight ranging from any of the minimum values described above to any of the maximum values described above. For example, in some embodiments, the polymer-functionalized polymerized hemoglobin can have a weight average molecular weight of from 500 kDa to 2000 kDa (e.g., from 700 kDa to 1500 kDa).

The polymer-functionalized polymerized hemoglobin can be substantially free of (e.g., can contain less than 5% by weight, less than 1% by weight, or less than 0.5% by weight) low-molecular weight hemoglobin species having a molecular weight of less than 100 kDa (e.g, substantially free of low-molecular weight hemoglobin species having a molecular weight of less than 250 kDa, or substantially free of low-molecular weight hemoglobin species having a molecular weight of less than 500 kDa).

In some embodiments, the polymer-functionalized polymerized hemoglobin exhibits an average hydrodynamic diameter of from 13 nm to 100 nm (e.g., from 13 nm to 50 nm, or from 13 nm to 30 nm), as measured by dynamic light scattering.

In some embodiments, the polymer-functionalized polymerized hemoglobin exhibits a zeta potential of from −20 mV to less than 0 mV (e.g., from −10 mV to less than 0 mV).

In some embodiments, the polymer-functionalized polymerized hemoglobin was polymerized in the T-state (tense quaternary state). In certain embodiments, the polymer-functionalized polymerized hemoglobin exhibits a P₅₀ of from 15 mm Hg to 40 mm Hg, a k_{off,O2} of from 10 s⁻¹ to 40 s⁻¹, or a combination thereof.

In other embodiments, the polymer-functionalized polymerized hemoglobin was polymerized in the R-state (relaxed quaternary state). In certain embodiments, the polymer-functionalized polymerized hemoglobin exhibits a P₅₀ of 1.0±0.5 mm Hg, a k_{off,O2} of from 7 s⁻¹ to 20 s⁻¹, or a combination thereof.

In some embodiments, compositions comprising the HBOCs described herein can compise a mixture comprising polymer-functionalized polymerized hemoglobin polymerized in the T-state (tense quaternary state) and polymer-functionalized polymerized hemoglobin polymerized in the R-state (relaxed quaternary state). By varying the relative amount of the polymer-functionalized polymerized hemoglobin polymerized in the T-state (tense quaternary state) and polymer-functionalized polymerized hemoglobin polymerized in the R-state (relaxed quaternary state) present in the compositions, the oxygen transport characteristics of the composition (e.g., P₅₀, k_{off,O2}, etc.) can be tuned.

### Methods of Making

Also provided are methods of producing a hemoglobin-based oxygen carrier (HBOC). These methods can comprise: (i) contacting hemoglobin with a multifunctional cross-linking agent to form a solution comprising polymerized hemoglobin; (ii) filtering the solution comprising polymerized hemoglobin by ultrafiltration (e.g., tangential flow filtration) against a first filtration membrane having a pore size that separates the polymerized hemoglobin from low-molecular weight hemoglobin species, reactants, and reaction byproducts, thereby forming a retentate fraction comprising the polymerized hemoglobin and a permeate fraction comprising impurities; (iii) covalently conjugating one or more polymers to the polymerized hemoglobin to form a solution comprising a polymer-functionalized polymerized hemoglobin; and (iv) filtering the solution comprising the polymer-functionalized polymerized hemoglobin by ultrafiltration (e.g., tangential flow filtration) against a second filtration membrane having a pore size that separates the polymer-functionalized polymerized hemoglobin from low-molecular weight impurities, reactants, and reaction byproducts, thereby forming a retentate fraction comprising the polymer-functionalized polymerized hemoglobin and a permeate fraction comprising impurities.

In some embodiments, step (i) can comprise deoxygenating the hemoglobin such that substantially all of the hemoglobin is in the T-state (tense quaternary state) prior to contacting the hemoglobin with the multifunctional cross-linking agent. In other embodiments, step (i) can comprise oxygenating the hemoglobin such that substantially all of the hemoglobin is in the R-state (relaxed quaternary state) prior to contacting the hemoglobin with the multifunctional cross-linking agent.

In some embodiments, the multifunctional cross-linking agent can comprise a dialdehyde, such as glutaraldehyde. The multifunctional cross-linking agent and the hemoglobin are present at a molar ratio of dialdehyde:hemoglobin of from 20:1 to 35:1.

In some examples, the hemoglobin utilized in step (i) can further comprise one or more antioxidant proteins which also react with the multifunctional cross-linking agent, thereby becoming co-polymerized with the hemoglobin. The one or more antioxidant proteins can comprise antioxidant proteins present in red blood cells, such as a peroxiredoxin (e.g., peroxiredoxin-1, -2, and/or -6), a superoxide dismutase, a catalase, or a combination thereof. In some examples, step (i) can be performed using a clarified red blood cell lysate which includes a mixture of hemoglobin, antioxidant proteins, and optionally one or more additional proteins found in red blood cells.

The first filtration membrane can be rated for removing solutes having a molecular weight less than the molecular weight of the polymerized hemoglobin. In some examples, the first filtration membrane is rated for removing solutes having a molecular weight of from 1 to 500 kDa, from 1 to 250 kDa, from 1 to 100 kDa, from 1 to 50 kDa, or from 1 to 10 kDa.

In some embodiments, step (ii) can further comprise filtering the solution comprising polymerized hemoglobin by ultrafiltration (e.g., tangential flow filtration) against a third filtration membrane having a pore size that separates the polymerized hemoglobin from high-molecular weight hemoglobin species, reactants, and reaction byproducts, thereby forming a permeate fraction comprising the polymerized hemoglobin and a retentate fraction comprising impurities.

In some examples, the third filtration membrane can be rated for retaining solutes having a molecular weight greater than the molecular weight of the polymerized hemoglobin. For example, the third filtration membrane can be rated for retaining solutes having a molecular weight of at least 500 kDa, at least 750 kDa, at least 1000 kDa, or more. In certain embodiments, the third filtration membrane can have a pore size of at least about 0.1 μm, such as a pore size of about 0.2 μm.

In some embodiments, following step (ii), substantially all of the polymerized hemoglobin has a molecular weight of at least 100 kDa, such as at least 250 kDa or at least 500 kDa. For example, in certain embodiments, following step (ii), substantially all of the polymerized hemoglobin has a molecular weight of from 100 kDa to 10,000 kDa, such as from 100 kDa to 500 kDa, from 100 kDa to 750 kDa, from 100 kDa to 1,000 kDa, from 100 kDa to 5,000 kDa, from 250 kDa to 500 kDa, from 250 kDa to 750 kDa, from 250 kDa to 1,000 kDa, from 250 kDa to 5,000 kDa, 250 kDa to 10,000 kDa, from 500 kDa to 750 kDa, from 500 kDa to 1,000 kDa, from 500 kDa to 5,000 kDa, 500 kDa to 10,000 kDa, from 750 kDa to 1,000 kDa, or from 750 kDa to 5,000 kDa, or from 750 kDa to 10,000 kDa.

Step (iii) can comprise covalently conjugating one or more polyalkylene oxides polymers, such as one or more polyethylene glycol (PEG) polymers, to the polymerized hemoglobin to form a solution comprising a polyalkylene oxide (PAO)-functionalized polymerized hemoglobin, such as a polyethylene glycol (PEG)-functionalized polymerized hemoglobin. For example, in some embodiments, step (iii) can comprise contacting the polymerized hemoglobin with a thiolating reagent (e.g., 2-Iminothiolane, Traut's reagent) and a malemidyl-activated PAO, such as a malemidyl-activated polyethylene glycol (Mal-PEG).

The second filtration membrane can be rated for removing solutes having a molecular weight less than the molecular weight of the PAO-functionalized polymerized hemoglobin. In some examples, the second filtration membrane is rated for removing solutes having a molecular weight of from 1 to 750 kDa, from 1 to 500 kDa, from 1 to 250 kDa, or from 1 to 100 kDa.

In connection with the methods described above, ultrafiltration can comprise direct-flow filtration (DFF), cross-flow or tangential-flow filtration (TFF), or a combination thereof. In certain embodiments, the ultrafiltration can comprise tangential-flow filtration (TFF).

The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler F R. Tangential flow filtration for processing cells, proteins, and other biological components. ASM News 1984; 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone. In some cases, the filtration membrane can be rated for retaining solutes having a molecular weight of from 1 to 750 kDa, such as from 1 to 500 kDa, from 1 to 250 kDa, from 1 to 100 kDa, or from 1 to 50 kDa.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

### Methods of Use

The HBOCs and compositions described herein can be used as blood substitutes or additives to blood or other solutions to facilitate oxygen transport. As such, these compositions can be administered to subjects suffering with a wide range of diseases, disorders, and conditions.

The HBOCs and compositions described herein can exhibit reversible oxygen binding capacities which provide for oxygen transport properties. The HBOCs and compositions described herein can demonstrate good loading and unloading characteristics in usage which can correlate to having an oxygen-hemoglobin dissociation curve (Po) similar to whole blood. The HBOCs and compositions described herein can show a high affinity for binding oxygen in the capillaries through the lungs and then adequately release oxygen to the tissues in the body.

Insofar as the physiological properties are concerned, the HBOCs and compositions described herein can not cause vasoconstriction, renal toxicity, hemoglobinurea and other problems implicated with intravenous administration of known HBOCs. Upon intravenous administration of the HBOCs and compositions described herein, no appreciable decrease in urine production, no appreciable decrease in glomerular filtration rate, no appreciable extravasation into the peritoneal cavity and/or no appreciable change in the color of urine produced can be observed in the subject.

In some embodiments, the HBOCs and compositions described herein can find application in the treatment of trauma, myocardial infarction, stroke, acute anemia and oxygen deficiency disorders such as hypoxemia, hypoxia or end stage hypoxia due to impairment or failure of the lung to fully oxygenate blood. The HBOCs and compositions described herein can also be used to diseases or medical conditions requiring a resuscitative fluid (e.g . . . trauma, specifically hemorrhagic shock), intravascular volume expander or exchange transfusion. In addition to medical treatment, the HBOCs and compositions described herein can also be used to preserve organs for transplantation.

In some cases, the HBOCs and compositions described herein can be administered to a subject to treat a loss of blood due to injury, hemolytic anemia, equine infectious anemia, feline infectious anemia, a bacterial infection, Factor IV fragmentation, hypersplenation, splenomegaly, hemorrhagic syndrome, hypoplastic anemia, aplastic anemia, idiopathic immune hemadytic conditions, iron deficiency, isoimmune hemdytic anemia, microangiopathic hemolytic anemia, parasitism, or any combination thereof

HBOCs and compositions described herein can also be used in a variety of applications where a rapid restoration of O₂ levels or an increased O₂ level or a replacement of O₂ levels is clinically indicated, such as the following:
Trauma. An acute loss of whole blood can result in a fluid shift from the interstitial and intracellular spaces to replace the lost volume of blood while shunting of blood away from the low priority organs including the skin and gut. Shunting of blood away from organs reduces and sometimes eliminates O₂ levels in these organs and results in progressive tissue death. Rapid restoration of O₂ levels is contemplated as perhaps resulting in a significantly better salvage of tissues in patients suffering such acute blood loss.
Ischemia. In ischemia, a particular organ (or organs) is “starved” for oxygen. Small sections of the organ, known as infarcts, begin to die as a result of the lack of O₂. Rapid restoration of O₂ levels is critical is stemming infarct formation in critical tissues. Conditions resulting in ischemia include heart attack, stroke, or cerbrovascular trauma.
Hemodilution: In this clinical application, a blood substitute is required to replace blood that is removed pre-operatively. It is contemplated that the patient blood removal occurs to prevent a requirement for allogeneic transfusions post-operatively. In this application, the blood substitute is administered to replace (or substitute for) the O₂ levels of the removed autologous blood. This permits the use of the removed autologous blood for necessary transfusions during and after surgery. One such surgery requiring pre-operative blood removal would be a cardiopulmonary bypass procedure.
Septic Shock. In overwhelming sepsis, some patients may become hypertensive in spite of massive fluid therapy and treatment with vasocontrictor agents. In this instance, the overproduction of nitric oxide (NO) results in the lowered blood pressure. Therefore, hemoglobin is close to an ideal agent for treatment of these patients because hemoglobin binds NO with an avidity that parallels O₂.
Cancer. Delivery of O₂ to the hypoxic inner core of a tumor mass increases its sensitivity to radiotherapy and chemotherapy. Because the microvasculature of a tumor is unlike that of other tissues, sensitization through increasing Oz levels requires O₂ be unloaded within the hypoxic core. In other words, the P₅₀ should be very low to prevent early unloading of the O₂, increasing the O₂ levels, to insure optimal sensitization of the tumor to subsepuent radiation and chemotherapy treatments.
Chronic anemia. In these patients, replacement of lost or metabolized hemoglobin is compromised or completely absent. It is contemplated that the blood substitute must effectively replace or increase the reduced O₂ levels in the patient.
Sickle cell anemia. In sickle cell anemia, the patient is debilitated by a loss of O₂ levels that occurs during the sickling process as well as a very high red blood cell turnover rate. The sickling process is a function of P_{O2} where the lower the P_{O2}, the greater the sickling rate. It is contemplated that the ideal blood substitute would restore patient O₂ levels to within a normal range during a sickling crisis.
Cardioplegia. In certain cardiac surgical procedures, the heart is stopped by appropriate electrocyte solutions and reducing patient temperature. Reduction of the temperature will significantly reduce the P₅₀, possibly preventing unloading of O₂ under any ordinary physiological conditions. Replacement of O₂ levels is contemplated as potentially reducing tissue damage and death during such procedures.
Hypoxia. Soldiers, altitude dwellers, and world-class athletes under extreme conditions may suffer reduced O₂ levels because extraction of O₂ from air in the lung is limited. The limited O₂ extraction further limits O₂ transport. It is contemplated that a blood substitute could replace or increase the Oz levels in such individuals.
Organ Perfusion. During the time an organ is maintained ex vivo, maintaining O₂ content is essential to preserving structural and cellular intergrity and minimizing infarct formation. It is contemplated that a blood substitute would sustain the O₂ requirements for such an organ.
Cell Culture. This requirement is virtually identical to that of organ perfusion, except that the rate of O₂ consumption may be higher.
Hematopoiesis. It is contemplated that the blood substitute serves as a source for heme and iron for use in the synthesis of new hemoglobin during hematopoiesis.
HBOCs and compositions described herein can also be used in non-humans, including domestic animals such as livestock and companion animals (e.g, dogs, cats, horses, birds, reptiles), as well as other animals in aquaria, zoos, oceanaria, and other facilities that house animals.

### EXAMPLES

The invention will be described in greater detail by way of specific examples. The following examples are offered for illustrative purposes and are not intended to limit the invention in any manner. Those of skill in the art will readily recognize a variety of non-critical parameters which can be changed or modified to yield essentially the same results.

### Example 1: Tangential Flow Filtration Facilitated Fractionation and PEGylation of Low and High Molecular Weight Polymerized Bovine Hemoglobins and their Biophysical Properties

### Summary

Various types of hemoglobin (Hb)-based oxygen carriers (HBOCs) have been developed as red blood cell substitutes for treating blood loss when blood is not available. Among those HBOCs, glutaraldehyde polymerized Hbs have attracted significant attention due to their facile synthetic route, and ability to expand the blood volume and deliver oxygen. HEMOPURE®, OXYGLOBIN®, and POLYHEME® are the most well-known commercially developed glutaraldehyde polymerized Hbs. Unfortunately, only OXYGLOBIN® was approved by the FDA for veterinary use in the U.S., while HEMOPURE® and POLYHEME® failed phase III clinical trials due to their ability to extravasate from the blood volume into the tissue space which facilitated nitric oxide scavenging and tissue deposition of iron, which elicited vasoconstriction, hypertension and oxidative tissue injury. Fortunately, conjugation of poly (ethylene glycol) (PEG) on the surface of Hb can reduce the vasoactivity of Hb by creating a hydration layer surrounding the Hb molecule, which increases its hydrodynamic diameter and reduces tissue extravasation. Several commercial PEGylated Hbs (MP4®, SANGUINATE®, Euro-PEG-Hb) have been developed for clinical use with longer circulatory half-life and improved safety compared to Hb. However, all of these commercial products exhibited relatively high oxygen affinity compared to Hb, which limited their clinical use.

In order to dually address the limitations of prior generations of polymerized and PEGylated Hbs, this current study describes the PEGylation of polymerized bovine Hb (PEG-PolybHb) in both the tense (T) and relaxed (R) quaternary state via thiol-maleimide chemistry to produce an HBOC with low oxygen affinity. The biophysical properties of PEG-PolybHb were measured and compared with those of commercial polymerized and PEGylated HBOCs. T-state PEG-PolybHb possessed higher hydrodynamic volume and P₅₀ than previous generations of commercial PEGylated Hbs. Both T- and R-state PEG-PolybHb exhibited significantly lower haptoglobin binding rates than the precursor PolybHb, indicating potentially reduced clearance by CD163+ monocytes and macrophages. Thus, PEG-PolybHb is expected to function as a promising HBOC due to its low oxygen affinity and enhanced stealth properties afforded by the PEG hydration shell.

### Introduction

Hemoglobin-based oxygen carriers (HBOCs) are of interest as red blood cell (RBC) substitutes to treat blood loss when blood is not available. A variety of strategies have been used to synthesize/manufacture HBOCs such as hemoglobin (Hb) polymerization with chemical cross-linking agents, surface conjugation of Hb with poly(ethylene glycol)and liposome encapsulation of Hb. Among these strategies, glutaraldehyde cross-linking of Hb to form polymerized Hb (PolyHb) is the most attractive synthetic route due to its low costs and scalable production¹. Several commercial glutaraldehyde PolyHbs have been developed, including HEMOPURE®, OXYGLOBIN®, and POLYHEME®. Of these, only OXYGLOBIN® was approved by the FDA for veterinary use. Unfortunately, HEMOPURE® and POLYHEME® elicited vasoconstriction, systemic hypertension and oxidative tissue injury during phase III clinical trials, which hindered their commercial development. These side-effects have been attributed to the presence of low molecular weight (MW) Hb species in formulations of HEMOPURE® and POLYHEME®, which can extravasate through the blood vessel wall into the tissue space, where the Hb species can scavenge nitric oxide (NO) and deposit redox active iron, which induces vasoconstriction, systemic hypertension and oxidative tissue injury. For example, POLYHEME® extravasation into the perivascular space induced myocardial infarction, mostly a consequence of ROS formation which damaged tissue. Removing low MW Hb species (<500 kDa) from HBOC formulations offers a possible strategy for avoiding these side-effects. Towards this end, we have produced high MW (>500 kDa) polymerized bovine Hb (PolybHb) via a controlled polymerization approach, which produces PolybHb formulations with very little low MW Hb species in solution. By appropriately engineering PolybHb molecular diameter, it was possible to reduce PolybHb tissue extravasation and renal toxicity. For the most optimal PolybHb size, the half-life was ˜30 hours, but the PolybHb was still able to weakly bind the plasma Hb scavenger protein haptoglobin (Hp) thereby facilitating clearance via CD163+ monocytes and macrophages. To potentially reduce PolybHb clearance via CD163-mediated endocytosis and possibly extend PolybHb circulatory half-life, the biophysical properties of poly(ethylene glycol) (PEG) surface conjugated low and high MW PolybHb fractions in the tense (T) and relaxed (R) quaternary states were investigated.

Conjugation of PEG on the surface of Hb (PEGylation of Hb) can reduce the vasoactivity of Hb, prevent tissue extravasation and the associated side-effects of Hb, and increase circulatory half-life compared to Hb. In addition, PEGylated Hbs (PEG-Hbs) also exhibited high colloidal osmotic pressure (COP), which imparts favorable plasma expansion properties when the material is transfused into the blood stream. All of these properties allow PEG-Hb to serve as a promising RBC substitute. HEMOSPAN® (MP4, Sangart Inc., San Diego, CA, USA) is a commercial PEG-Hb that was produced via site-specific thiolation of lysine residues on the surface of human Hb (HbA) with iminothiolane followed by conjugation with maleimide-PEG chains. HEMOSPAN® exhibits a very high oxygen affinity (P₅₀=5˜6 mm Hg) in comparison to HbA (P₅₀=12 mm Hg). SANGUINATE® is an example of a PEGylated bovine Hb, which has a high oxygen affinity (P₅₀=14.0 mm Hg) in comparison to native bovine Hb (P₅₀=28 mm Hg). The significantly increased oxygen affinity of HEMOSPAN® and SANGUINATE® facilitates oxygen delivery under hypoxic conditions. It is also known that PEGylation of Hb promotes dimerization of the Hb tetramer (α₂β₂) into αβ dimers, and increases the Hb autoxidation rate which leads to rapid methemoglobin (metHb) formation and loss of oxygen carrying capacity. Herein, we combine Hb polymerization and subsequent PEGylation to increase the molecular diameter of PolybHb molecules fractionated into low and high MW fractions via tangential flow filtration (TFF).

### Materials and Methods

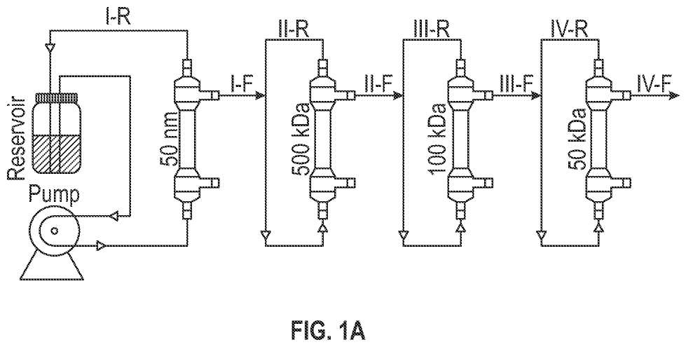

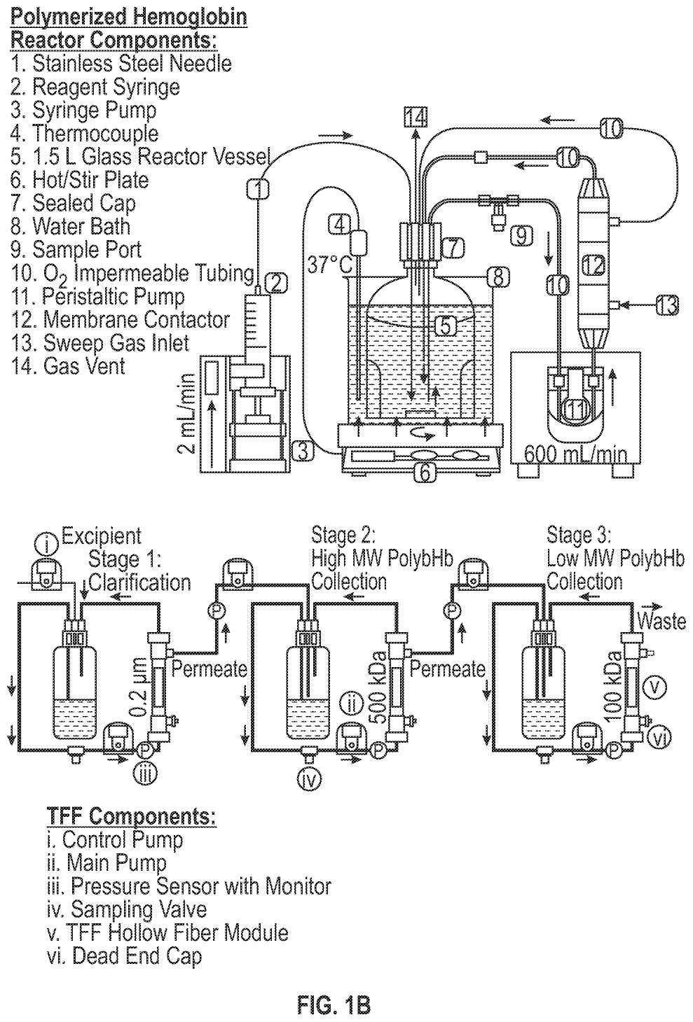

Hb Purification. Bovine Hb (bHb) was purified via TFF as described previously. Briefly, a KrosFlo Research II TFF system configured with various HF filter modules was used to purify Hb (FIG. 1A). Four hollow filter (HF) filter modules were selected for the multistage purification of Hb and consisted of 50 nm, 500 kDa, 100 kDa, and 50 kDa (MWCO) membranes. Prior to use, the four HF filter modules were thoroughly rinsed with deionized H₂O. Each of the four HF filter modules was tested to make sure that their structural integrity was not compromised (i.e., to ensure that the individual HFs comprising the HF cartridge were not broken).

Four HF cartridges were used to purify Hb from impurities that were present in the RBC lysate (FIG. 1A). At each filtration stage, filtrate was collected and retentate recycled to maximize the yield of Hb. However, at stage III, the filtrate from stage II was diafiltrated with 1 L of PB. The entire purification process was conducted in a biological safety cabinet, and the filtrate and retentate reservoirs were kept on ice. Fifty nanometer, 500 kDa, 100 kDa, and 50 kDa HF cartridges were used in stages I, IL, III, and IV of the Hb purification process, respectively. The permeate flow rate and transmembrane pressure difference were measured at the beginning and end of the purification process for each stage. After each stage, 10 mL of sample was taken from the filtrate and retentate for subsequent analysis. The initial stage I filtration process started with 2,000 mL of RBC lysate. After each run, the HF module, associated tubing, and reservoir bottles were sanitized with 0.5 M NaOH solution to remove any protein adhering to the HF membrane and to degrade any endotoxin that may be present. After going through the sanitizing process, HF modules were stored in 0.01% SDS solution with 0.02% NaN₃ (to inhibit bacterial growth). Before use, the HF module, associated tubing, and reservoir bottles were washed extensively with deionized water.

For the first two stages (I and II), the majority of protein passed through the HF membrane into the filtrate reservoir. During stage III, most of the Hb was retained in the retentate stream. This fraction was used as bHb for future studies.

Total Hb and Methemoglobin (MetHb) Levels. Total Hb and metHb concentrations were determined using the cyanmethemoglobin method. Spectrophotometric absorbance measurements were obtained using a HP 8452A diode array spectrophotometer (Olis, Bogart, GA).

Synthesis and Fractionation of PolybHb. Thirty grams of bHb was diluted with phosphate buffered saline (PBS, 0.1 M, pH 7.4) to yield 1.5 L of a ˜20 mg/ml bHb solution. The solution was then transferred into an airtight, amber-tinted reactor vessel as shown in FIG. 1B. In this example, PolybHbs (i.e., T-state PolybHb 30:1[molar ratio of glutaraldehyde to bHb] and R-state PolybHb 25:1 [molar ratio of glutaraldehyde to bHb]) were synthesized in two distinct quaternary states for subsequent TFF fractionation into a <500 kDa low MW (LMW) PolybHb fraction and a >500 kDa high MW (HMW) PolybHb fraction. T-state bHb was generated by completely deoxygenating the bHb solution using a 3M MiniModule gas/liquid exchange module (Maplewood, MN) followed by a bolus injection of sodium dithionite.

The polymerization reaction was initiated when the partial pressure of O₂ in solution (pO₂) reached a value of 0.0 mm Hg. Continuous purging of the reactor headspace with N₂ was performed throughout the T-state polymerization process. While for R-state PolybHb 25:1, polymerization was initiated when the pO₂ reached above 745 mm Hg to guarantee complete oxygenation of bHb. Fifty mL of glutaraldehyde at the appropriate molar ratio of glutaraldehyde to bHb was added to the bHb solution at a flow rate of 2 mL/min followed by a 2-hour reaction at 37° C. with continuous stirring. NaCNBH₃ was injected into the reactor to quench the reaction.

After synthesis, both T- and R-state PolybHb were sterile filtered via TFF on a 0.2 μm hollow fiber (HF) module. Both HMW (>500 kDa) T-state PolybHb 30:1 and R-state PolybHb 25:1 were diafiltered and concentrated on a 500 kDa HF module, while the LMW species (<500 kDa, permeate of 500 kDa HF module) was diafiltered on a 100 kDa HF module. The rfactionated PolybHb was buffer exchanged into PBS (0.1 M, pH 7.4) for subsequent PEGylation.

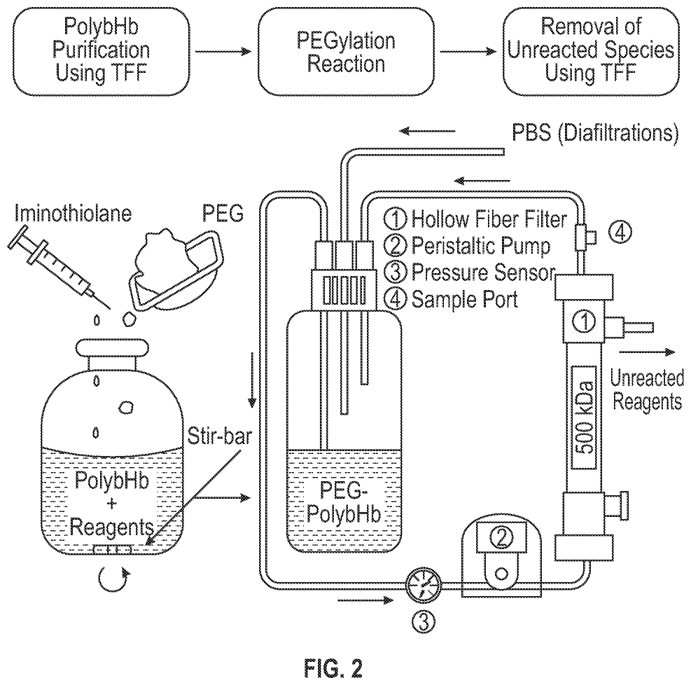

PEGylation of PolybHb. Briefly, the PolybHb fraction was thawed at 4° C. and diluted to ˜5 mg/mL in PBS. T- and R-PolybHb were incubated with 2×molar excess of 2-iminothiolane hydrochloride (IT, Fisher Scientific, Pittsburgh, PA) and a 6×molar excess mPEG-maleimide, MW 5000 g/mol (Laysan Bio, Alabama, USA). The reaction mixture was incubated at 4° C. for 16 hours or overnight with constant stirring (FIG. 2). After the reaction was completed, unreacted reagents were removed by diafiltration using PBS over a 500 kDa HF module. Ten constant volume diafiltrations were carried out for adequate removal of reagents and buffer exchange into PBS. The product was concentrated to ˜5 mg/mL and stored at −80° C. for future use.

Quaternary Structure (SEC-HPLC). Unmodified bHb, T-state and R-state PolybHbs and PEG-PolybHbs were separated on an analytical Acclaim SEC-1000 (4.6×300 mm) column (Thermo Fisher Scientific, Waltham, MA). The mobile phase consisted of 50 mM sodium phosphate buffer (PB) at pH 7.4. Chromeleon 7 software was used to control HPLC parameters such as flow rate (0.35 mL/min), UV-visible absorbance detection (280 nm [to detect total protein] and 413 nm [to detect heme]). All samples were filtered through 0.2 μm syringe filters before size exclusion (SEC) HPLC analysis.

Hydrodynamic Diameter. The hydrodynamic diameter of bHb, PolybHbs and PEG-PolybHbs were measured using a BI-200SM goniometer (Brookhaven Instruments Corp., Holtsville, NY) at an angle of 900 and wavelength of 637 nm. Protein samples were diluted to ˜0.5-1 mg/mL concentration in deionized (DI) water. The hydrodynamic diameter was obtained by using average values from the non-linear least squared (NNLS) algorithm in the instrument software.

Oxygen (O₂) Equilibrium Curves. O₂ equilibrium curves (OECs) for bHb, PolybHbs and PEG-PolybHbs were measured using a Hemox Analyzer (TCS Scientific Corp., New Hope, PA). Protein samples were diluted to ˜60 μM (heme basis) in 5 mL Hemox buffer (pH 7.4) with 20 μL of Additive A, 20 μL of Additive B, and 20 μL of antifoaming solution (TCS Scientific). The temperature was maintained at 37.0±0.1° C. The Hill equation was used to fit the OEC data, and the P₅₀ (partial pressure of Oz at which the Hb is half saturated with O₂) and n (cooperativity coefficient) values were regressed from the curve fits.

Auto-Oxidation. Protein samples were diluted to 60 μM (heme basis) in PBS (0.1 M, pH 7.4). A UV-visible spectrophotometer attached to a recirculating water bath was used to monitor the absorbance spectra from 300-700 nm at 37° C. every 30 mins for 24 hours. The auto-oxidation rate constant was determined by fitting exponential decay kinetics to the disappearance of oxygenated Hb species in solution.

Rapid Deoxygenation Kinetics. Protein samples were diluted to 12.5 μM (heme basis) in PBS (0.1 M, pH 7.4). Deoxygenated buffer was prepared by adding 1.5 mg/mL of sodium dithionite to PBS bubbled under N₂ for 20-30 mins. Deoxygenated buffer and oxygenated protein samples were mixed rapidly in a microvolume stopped-flow spectrophotometer (Applied Photophysics Ltd., Surrey, United Kingdom) and the absorbance was monitored at 437.5 nm. An exponential decay function was fit to the data and the rate constant for O₂ dissociation (k_{off,O2}) was regressed for each sample.

Haptoglobin Binding Kinetics. The kinetics of haptoglobin (Hp) binding to bHb/PolybHb/PEG-PolybHb was measured. The reaction between Hp and bHb/PolybHb/PEG-PolybHb was monitored by stopped flow fluorescence spectrometry by excitation at 285 nm and monitoring the fluorescence emission at 310 nm. The pseudo first order Hp binding rate constant was calculated by fitting the fluorescence intensity to a mono-exponential equation. The pseudo first-order rate constant was then used to determine the bimolecular rate constant via linear regression with the PolybHb/PEG-PolybHb concentration as the dependent variable.

Statistical Analysis. In this example, all statistical analysis was performed using a t-test, and a p value of <0.05 was considered significant.

### Results and Discussion

Table 1 displays the biophysical properties of T- and R-state PolybHbs and PEG-PolybHbs for both LMW and HMW species. The effect of bHb quaternary state and size fractionated PolybHb. MW on the biophysical properties of PEG-PolybHbs was studied by comparing molecular diameter, zeta potential, oxygen equilibria, oxygen offloading rate constant, auto-oxidation rate constant, and Hp binding rate constant to unmodified bHb and size fractionated PolybHb.

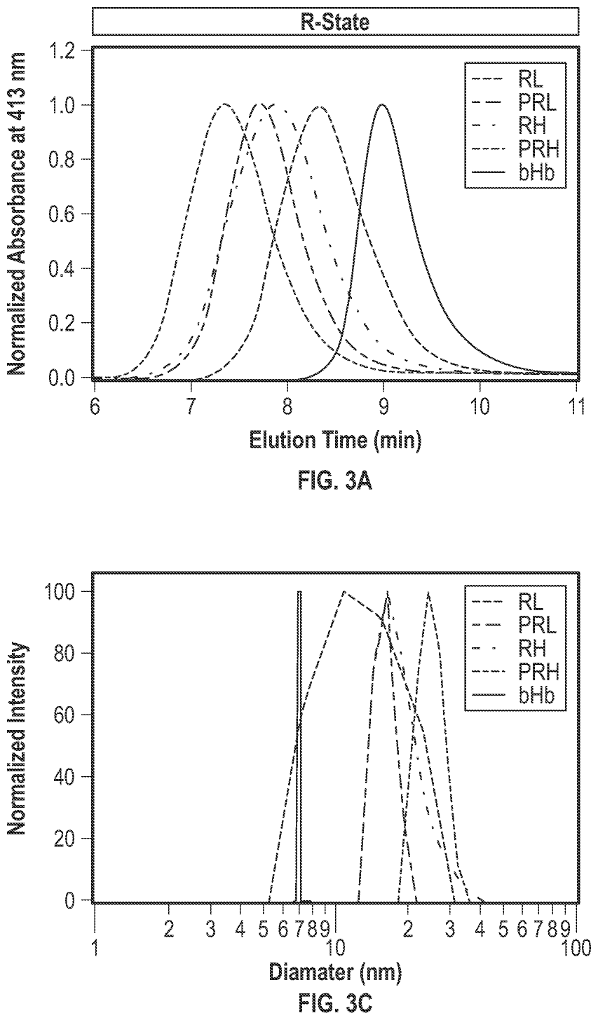

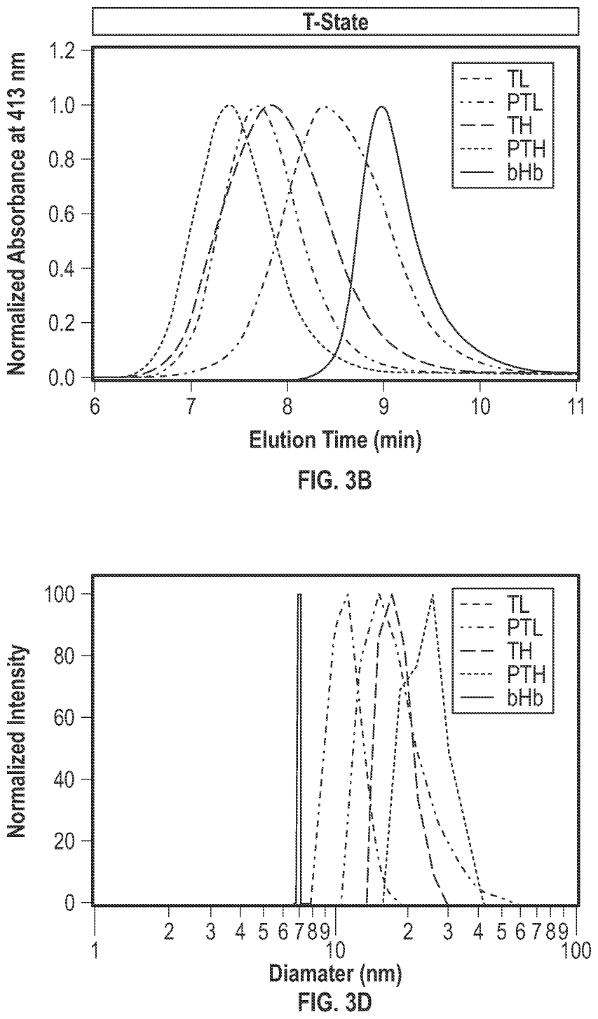

Size and Molecular Weight. T- and R-state PolybHbs and PEG-PolybHbs were analyzed using SEC-HPLC to determine MW changes upon PEGylation (FIG. 3A-3B). MW estimates were performed for all species using the standard MW calibration curve that is associated with the SEC-HPLC column. Unmodified bHb eluted at ˜9.1 mins, whereas all PolybHbs eluted at earlier times signifying an increase in bHb size upon polymerization. After PEGylation of the respective PolybHbs, it was observed that the PEGylated PolybHb species had a left shift in the elution chromatogram compared to the precusor PolybHb confirming successful PEGylation of the precursor PolybHb. For example, LMW R-state PolybHb (RL) eluted at ˜8.4 mins whereas its PEGylated derivative (PRL) eluted at ˜7.7 mins, indicative of an increase in MW and successful PEGylation. A similar left shift was observed for all T- and R-state PolybHb species after PEGylation. It was also observed that PEGylated PolybHb species displayed a narrower SEC-HPLC peak compared to their PolybHb precursor. PEG chains are known to increase the hydration radius of molecules, and hence increase the apparent MW in the SEC-HPLC chromatograms. However, PolybHb molecules already display a wide MW distribution (LMW species ˜100-500 kDa and HMW species ˜500 kDa-0.2 μm) due to the TFF size-based separation technique employed to prepare the LMW and HMW fractions. Furthermore, it was also observed that PEGylated LMW PolybHb had a larger apparent MW than unPEGylated HMW PolybHb. Compared to previous commercial HBOCs such as HEMOPURE® (MW ˜250 kDa), POLYHEME® (MW ˜150 kDa) and OXYGLOBIN® (MW ˜200 kDa), the materials

TABLE 1
Biophysical properties of LMW and HMW PEG-PolybHbs/PolybHbs in both the T-state and R-state. In total, 4 different
types of PolybHbs were synthesized and fractionated for subsequent PEGylation, this includes both HMW and LMW
T-state PolybHb 30:1 (TH and TL, respectively), and HMW and LMW R-state PolybHb 25:1 (RH and RL, respectively).
Their PEGylated derivatives correspond to the following abbreviations PTH, PTL, PRH, and PRL, respectively.

| Parameter | bHb | RL | PRL | RH | PRH | TL | PTL | TH | PTH |
|---|---|---|---|---|---|---|---|---|---|
| P₅₀ (mm Hg) | 26.3 ± | 1.6 ± | 1.0 ± | 1.7 ± | 1.1 ± | 40.8 ± | 20.2 ± | 35.1 ± | 20.1 ± |
| | 0.5 | 0.1 | 0.1 | 0.1 | 0.1 | 1.3 | 0.7 | 0.3 | 0.3 |
| Cooperativity | 2.50 ± | 0.84 ± | 0.84 ± | 1.03 ± | 0.70 ± | 0.99 ± | 1.00 ± | 0.99 ± | 0.91 ± |
| (n) | 0.10 | 0.06 | 0.04 | 0.03 | 0.02 | 0.01 | 0.01 | 0.03 | 0.02 |
| MW (kDa) | 65 | 303 | 768 | 503 | 1388 | 216 | 822 | 586 | 1460 |
| D_{off} (nm) | 6.92 ± | 10.7 ± | 16.3 ± | 18.9 ± | 24.4 ± | 11.2 ± | 15.3 ± | 17.3 ± | 25.7 ± |
| | 0.3 | 4.3 | 2.5 | 2.9 | 2.4 | 1.5 | 2.8 | 2.4 | 3.9 |
| PDI | 0.1 | 0.8 | 0.3 | 0.3 | 0.2 | 0.2 | 0.3 | 0.3 | 0.4 |
| Zeta potential | −21.2 ± | −13.5 ± | −4.35 ± | −11.1 ± | −3.10 ± | −17.7 ± | −2.16 ± | −22.5 ± | −7.29 ± |
| (mV) | 2.51 | 0.85 | 0.36 | 0.59 | 1.87 | 1.42 | 1.27 | 1.27 | 0.89 |
| k_{off, O2} (s⁻¹) | 45.86 ± | 19.64 ± | 15.89 ± | 16.78 ± | 9.84 ± | 39.96 ± | 25.93 ± | 30.94 ± | 19.95 ± |
| | 1.00 | 0.07 | 1.05 | 1.06 | 0.17 | 3.45 | 1.59 | 3.43 | 0.72 |
| k_{Hb-Hp} (μM⁻¹s⁻¹) | 0.1491 | 0.0070 | 0.0022 | 0.0080 | 0.0022 | 0.0185 | 0.0023 | 0.0201 | 0.0021 |
| k_{ox, fast} (h⁻¹) | 0.021 | 0.0150 | 0.0219 | 0.0076 | 0.0075 | 0.0096 | 0.0117 | 0.0186 | 0.0245 |
| K_{ox, slow} (h⁻¹) | 0.021 | 0.0067 | 0.0115 | 0.0057 | 0.0087 | 0.0048 | 0.0084 | 0.0082 | 0.0109 |
| MetHb level (%) | 1.0 ± | 1.9 ± | 4.6 ± | 1.9 ± | 4.8 ± | 1.9 ± | 5.5 ± | 1.9 ± | 6.9 ± |
| | 03 | 0.5 | 0.4 | 0.5 | 0.2 | 0.1 | 0.2 | 0.1 | 1.0 |

synthesized in this example had considerably higher MW, and hence should not extravasate into the tissue space and should reduce the side-effects associated with these commercial HBOCs. Similarly, as compared to previous commercial PEGylated HBOCs such as HEMOSPAN® and SANGUINATE®, which had MW ˜100 kDa, these materials have much higher MW, and because they are cross-linked should not form αβ dimers in solution.

To further confirm successful PEGylation of PolybHb, DLS analysis was performed on all materials (FIG. 3C-3D). As compared to bHb which had a diameter of 6.9 nm, all PolybHbs (LMW and HMW) had larger diameters ranging from 10.7-18.9 nm. Furthermore, after PEGylating LMW PolybHbs, the hydrodynamic diameter increased from 10.7 to 16.3 nm for R-state PolybHb and 11.2 to 15.3 nm for T-state PolybHb further confirming the SEC-HPLC results. A similar trend was observed for HMW PolybHb species, which increased in diameter from 18.9 to 24.4 nm for R-state PolybHb and 17.3 to 25.7 nm for T-state PolybHb after PEGylation. In comparison, previous commercial HBOCs such as HEMOPURE®, POLyHEME®, and OXYGLOBIN® had average diameters of 8.4, 7.9, and 8.1 nm respectively. The polydispersity of all PEGylated species remained <0.3 with the exception of R-state LMW PolybHb, which had a larger proportion of smaller MW species most likely due to the 100 kDa HF filter used during the diafiltration process.

Surface charge changes were assessed by measuring the zeta potential before and after PEGylation (Table 1). In general, polymerization reduced the zeta potential of bHb from −21 mV to ˜−15 mV after polymerization. After PEGylation, the zeta potential further decreased to ˜−4 mV owing to the neutrally charged PEG chains conjugated to the surface of the PolybHb molecules. This behavior has previously been observed for most PEGylated therapeutics³⁸. The partially negative zeta potential should prevent PEG-PolybHb from sticking to the blood vessel wall, red blood cells (RBCs), and other proteins in the bloodstream that tend to have partially negative (−10 to −20 mV) surface charge.

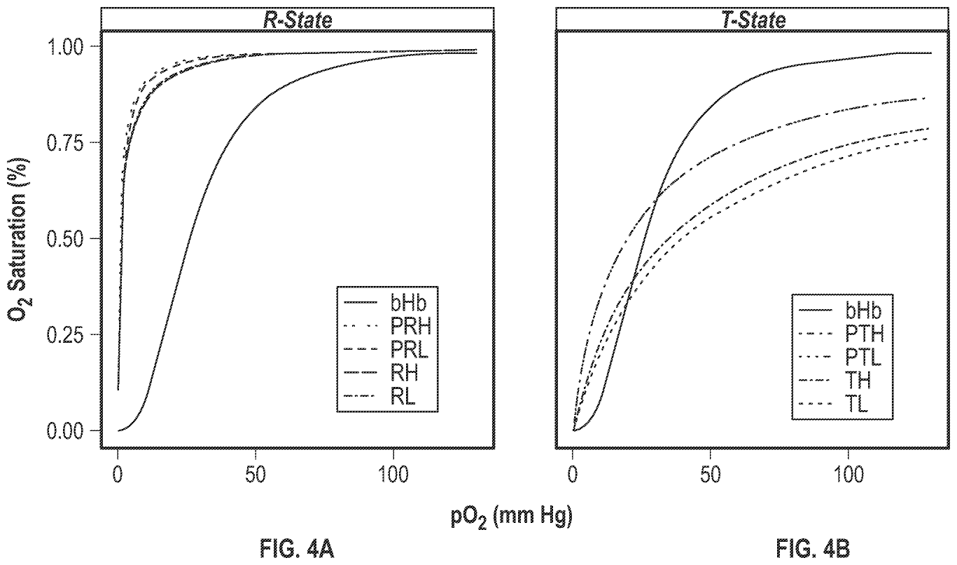

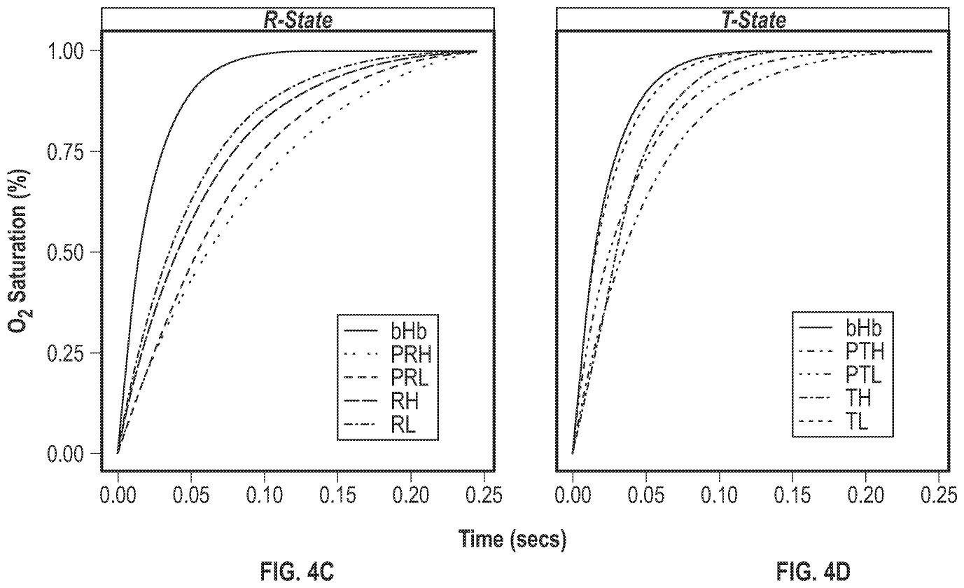

Oxygen Affinity and Offloading. FIGS. 4A and 4B show the OECs for bHb, T-state and R-state PolybHbs, and PEG-PolybHbs. The effect of PEGylation on the oxygen affinity (P₅₀) and Hill cooperativity coefficient (n) of PolybHbs was studied by fitting the OECs to the Hill equation to regress the P₅₀ and n. In general, a slight left shift in the OECs (higher O₂ affinity, i.e., lower P₅₀) was observed for all PEG-PolybHbs in comparison to the precursor PolybHbs, which is most likely attributed to chemical modification of the βCys93 residue of bHb, which increases the O₂ affinity. R-state PEGylated PolybHb 25:1 possessed a P₅₀ of 1.0±0.1 mm Hg and 1.1±0.1 mm Hg for HMW and LMW species, respectively. While for unmodified R-state PolybHb 25:1, a higher Po was observed for HMW (1.7±0.1 mm Hg) and LMW species (1.6±0.1 mm Hg). Similar differences were observed for T-state PolybHb 30:1. T-state PolybHb 30:1 possessed a P₅₀ of 35.1±0.3 mm Hg and 40.8±1.3 mm Hg for HMW and LMW species, respectively, which is similar to OXYGLOBIN® (˜38.4 mm Hg), HEMOLINK® (˜33.5 mm Hg), and HEMOPURE® (˜38 mm Hg), but higher than POLYHEME® (˜29 mm Hg). HMW and LMW PEGylated T-state PolybHb 30:1 (PTH and PTL) possessed a P₅₀ of 20.1±0.3 mm Hg and 20.2±0.7 mm Hg, respectively. The P₅₀ values of PEGylated T-state PolybHb represent a significant increase in comparison to commercial PEGylated HBOCs such as HEMOSPAN® (Sangart Inc, San Diego, CA) (˜5-6 mm Hg), SANGUINATE® (˜12 mm Hg), and Euro-PEG-Hb (˜14.13±0.36 mm Hg). All PEG-PolybHbs exhibited no cooperativity (n˜1.0), which is slightly lower than HEMOSPAN® (1.2) and Euro-PEG-Hb (1.48±0.10). The reduced cooperativity most likely is a consequence of polymerization combined with subsequent PEGylation, which restricts motion of the globins to facilitate cooperative O₂ binding.

The O₂ offloading kinetics of T-state and R-state PolybHbs and PEG-PolybHbs are shown in FIGS. 4C and 4D. In comparison to bHb, both PolybHbs and PEG-PolybHbs released O₂ slower than unmodified bHb. This could be due to O₂ diffusion limitations through the core of PolybHb and PEG-PolybHb nanoparticles. Before PEGylation, both HMW and LMW T-state PolybHb 30:1 (TH and TL, respectively) exhibited a higher O₂ offloading rate constant (k_{O2,off}) than R-state PolybHbs. This can be attributed to the increased O₂ affinity due to the physical constraints of bHb after polymerization in the R quaternary state. Interestingly, a difference between the k_{O2,off} of PEG-PolybHbs and PolybHbs is observed for both quaternary states. FIG. 4D displays the effect of the MW of T-state PolybHb/PEG-PolybHb on the O₂ offloading kinetics. TL (39.96±3.45 s⁻¹) exhibited a significantly higher k_{O2,off} than TH (30.94±3.43 s⁻¹), which is reflective of the larger diffusive barrier to O₂ transport due to the larger particle diameter of TH compared to TL. Both TL and TH exhibited a lower k_{O2,off} in comparison to OXYGLOBIN® (61.8±1.6 s⁻¹). Similar results were observed for RL (19.64±0.07 s⁻¹) and RH (16.78±1.06 s⁻¹) as well (FIG. 4C). Similar differences were also observed between the k_{O2,off} of T- and R-state PEG-PolybHb. Specifically, PRL (15.89±1.05 s⁻¹) and PTL (25.93±1.59 s⁻¹) exhibited a significantly higher k_{O2,off} in comparison to PRH (9.84±0.17 s⁻¹) and PTH (19.95±0.72 s⁻¹), indicating that the k_{O2,off} of PEG-PolybHb can be engineered by size fractionation based on MW. The lower values of k_{O2,off} for PEG-PolybHb compared to the precursor PolybHb can be explained by the larger diffusive barrier to O₂ transport due to the extra PEG hydration layer afforded by PEG-PolybHb compared to the precursor PolybHb.

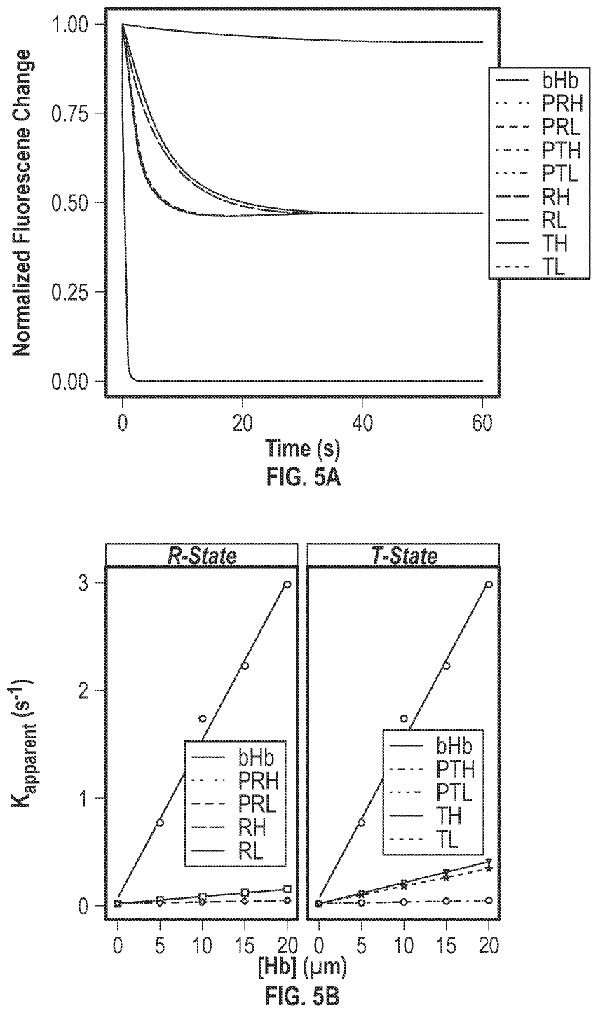

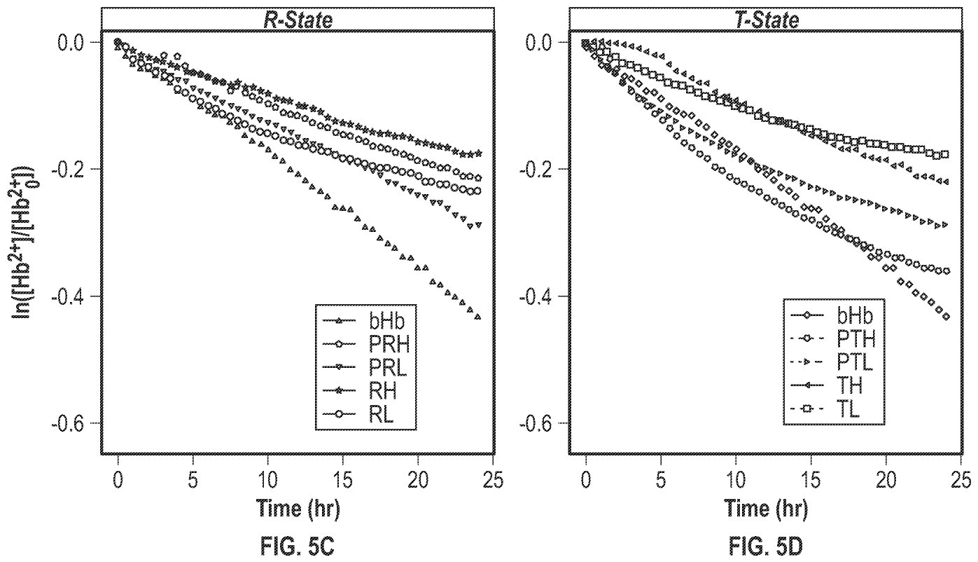

Haptoglobin Binding Kinetics. The potential clearance of PolybHb and PEG-PolybHb elicited by monocyte and macrophage CD163-mediated endocytosis was studied via measuring the Hp-binding kinetics with PolybHb/PEG-PolybHb. FIG. 5A displays the Hp binding kinetics, where all PEG-PolybHbs quenched a negligible amount of Hp, indicating potentially lower in vivo clearance via CD163-mediated endocytosis, which should exhibit a longer circulatory half-life in comparison to the corresponding PolybHb precursor. The pseudo first order binding rate constants was obtained by fitting the kinetic traces to a mono-exponential equation. A linear fit of pseudo first order binding rate constants at various concentrations of PolybHb/PEG-PolybHb yielded the second order Hp binding rate constant (k_{Hp-Hb}) as shown in FIG. 5B. Specifically, a much lower k_{Hp-Hb} was observed for LMW T-state PEG-PolybHb 30:1 (PTL) (0.0025 μM⁻¹ s⁻¹) in comparison to unmodified LMW T-state PolybHbs (0.0185 μM⁻¹ s⁻¹). Similar differences were also observed between LMW R-state PolybHb (0.0070 μM⁻¹ s⁻¹) and its PEGylated derivatives (0.0022 μM⁻¹ s⁻¹). The k_{Hp-Hb} for all PEG-PolybHbs were significantly reduced in comparison to the veterinary PolybHb product OXYGLOBIN® (0.011 μM⁻¹ s⁻¹)³⁷. The effect of the MW of the polymeric species on k_{Hp-Hb} was also evaluated in this example. Interestingly, such differences were negligible between the HMW and LMW PEG-PolybHbs, most likely due to the formation of the hydration layer surrounding the PolybHbs after PEGylation, which prevents binding of Hp. Therefore, the available Hb tetramers on the surface of PolybHb were much less exposed to Hp due to the steric hydration barrier created by the conjugated PEG chains.

Autoxidation Rate. FIG. 5C shows the auto-oxidation kinetics of bHb, PolybHbs, and PEG-PolybHbs. Two-phase auto-oxidation kinetics were observed for all PolybHbs. This two-phase behavior was also observed for all PEGylated derivatives, and mostly due to the different types of intramolecular cross-links (α-α, β-β and α-β) within the PolybHb molecule. For the fast-step auto-oxidation kinetics, both HMW (0.0245 hr⁻¹) and LMW (0.0117 hr⁻¹) T-state PEG-PolybHb yielded a slightly increased auto-oxidation rate constant in comparison to HMW (0.0186 hr⁻¹) and LMW (0.0096 hr⁻¹) T-state PolybHb. Similar results were observed for R-state PolybHb and its PEGylated derivatives as well. While for the slow-step auto-oxidation kinetics, the rate constant of all PEG-PolybHbs was higher than that of the corresponding PolybHbs. This could be a consequence of destabilizing effect caused by chemical modification of the βCys93 residue. Interestingly, in this example, the auto-oxidation rate constant of PEG-PolybHb was not dramatically increased in comparison to PolybHb likely due to the presence of inter- and intra-molecular cross-links after polymerization.

### CONCLUSION

In this example, we successfully synthesized T- and R-state LMW and HMW PolybHbs and surface conjugated these molecules with poly (ethylene glycol). Comprehensive biophysical characterization was performed to compare the size, MW, O₂ offloading kinetics, and Hp binding kinetics with other commercial polymerized and PEGylated HBOCs. PEG-PolybHb synthesized in this example showed a significant increase in size and MW along with higher oxygen affinity and a slightly increased auto-oxidation rate compared to their precursor PolybHb. The example presents a case for the use of PEG-PolybHb as a RBC substitute with potentially longer circulatory half-life, that should present limited risks related to vasoconstriction, systemic hypertension and oxidative tissue toxicity.

### ABBREVIATIONS

FDA Food and Drug Administration
HBOC Hemoglobin-based oxygen carrier
bHb Bovine hemoglobin
PolybHb Polymerized bovine hemoglobin
PEG-PolybHb PEGylated polymerized bovine hemoglobin
CD Circular dichroism
DI Deionized water
DLS Dynamic light scattering
Hb Hemoglobin
HF Hollow fiber
hHb Human hemoglobin
Hp Haptoglobin
k_{O2,off} O₂ dissociation rate constant
MetHb Methemoglobin
MW Molecular weight
n Hill coefficient
NO Nitric oxide
P₅₀ Partial pressure of O₂ at which 50% of the hemoglobin is saturated with O₂
PB Phosphate buffer
PBS Phosphate buffered saline
pO₂ Partial pressure of O₂
PolyHb Polymerized hemoglobin
R-State Relaxed quaternary state
T-State Tense quaternary state
RBC Red blood cell
SDS-PAGE Sodium dodecyl sulfate polyacrylamide gel electrophoresis
HPLC-SEC Size exclusion high performance liquid chromatography
TFF Tangential flow filtration

### REFERENCES

(1) Moore, E. E.; Cheng, A. M.; Moore, H. B.; Masuno, T.; Johnson, J. L. Hemoglobin-Based Oxygen Carriers in Trauma Care: Scientific Rationale for the US Multicenter Prehosptial Trial. World. Surg. 2006, 30 (7), 1247-1257. https://doi.org/10.1007/s00268-005-0499-6.
(2) Cabrales, P.; Sun, G.; Zhou, Y.; Harris, D. R.; Tsai, A. G.; Intaglietta, M.; Palmer, A. F. Effects of the Molecular Mass of Tense-State Polymerized Bovine Hemoglobin on Blood Pressure and Vasoconstriction. J. Appl. Physiol. 2009, 107(5), 1548-1558. https://doi.org/10.1152/japplphysiol.00622.2009.
(3) Buehler, P. W.; Zhou, Y., Cabrales, P.; Jia, Y.; Sun, G.; Harris, D. R.; Tsai, A. G.; Intaglietta, M.; Palmer, A. F. Synthesis, Biophysical Properties and Pharmacokinetics of Ultrahigh Molecular Weight Tense and Relaxed State Polymerized Bovine Hemoglobins. Biomaterials, 2010, 31 (13), 3723-3735. https://doi.org/10.1016fj.biomaterials.2010.01.072.
(4) Jahr, J. S.; Moallempour, M.; Lim, J. C. HBOC-201, Hemoglobin Glutamer-250 (Bovine), Hemopure (Registered Trademark) (Biopure Corporation). Expert Opin. Biol. Ther. 2008, 8 (9), 1425-1433.
(5) VANDEGRIFF, K. D.; YOUNG, M. A.; KEIPERT, P. E.; WINSLOW, R. M. The Safety Profile of Hemospan®: A New Oxygen Therapeutic Designed Using Maleimide Poly(Ethylene) Glycol Conjugation to Human Hemoglobin. Transfus. Altern. Transfus. Med. 2007, 9 (4), 213-225. https://doi.org/10.1111/j.1778-428X.2007.00083.x.
(6) Abuchowski, A. SANGUINATE (PEGylated Carboxyhemoglobin Bovine): Mechanism of Action and Clinical Update. Artif Organs 2017, 41 (4), 346-350. https://doi.org/10.1111/aor.12934.
(7) Rameez, S.; Guzman, N.; Banerjee, U.; Fontes, J.; Paulaitis, M. E.; Palmer, A. F.; Patel, R. P.; Honavar, J. Encapsulation of Hemoglobin inside Liposomes Surface Conjugated with Poly(Ethylene Glycol) Attenuates Their Reactions with Gaseous Ligands and Regulates Nitric Oxide Dependent Vasodilation. Biotechnol. Prog. 2012, 28 (3), 636-645. https://doi.org/10.1002/btpr.1532.
(8) Azuma, H.; Fujihara, M.; Sakai, H. Biocompatibility of Hemoglobin Vesicles, a Cellular-Type Artificial Oxygen Carrier, on Blood Cells and Plasma Proteins In Vitro and In Vivo. In Hemoglobin-Based Oxygen Carriers as Red Cell Substitutes and Oxygen Therapeutics; Springer Berlin Heidelberg: Berlin, Heidelberg, 2013; Vol. 9783642407, pp 385-397. https://doi.org/10.1007/978-3-642-40717-8_22.
(9) Rameez, S.; Banerjee, U.; Fontes, J.; Roth, A.; Palmer, A. F. Reactivity of Polymersome Encapsulated Hemoglobin with Physiologically Important Gaseous Ligands: Oxygen, Carbon Monoxide, and Nitric Oxide. Macromolecules 2012, 45 (5), 2385-2389. https://doi.org/10.1021/ma202739f.
(10) Ikeda, T.; Horinouchi, H.; Izumi, Y.; Sakai, H.; Kobayashi, K. Cellular-Type Hemoglobin-Based Oxygen Carrier as a Resuscitative Fluid for Hemorrhagic Shock: Acute and Long-Term Safety Evaluation Using Beagle Dogs. In Hemoglobin-Based Oxygen Carriers as Red Cell Substitutes and Oxygen Therapeutics; Springer Berlin Heidelberg: Berlin, Heidelberg, 2013; Vol. 9783642407, pp 501-524. https://doi.org/10.1007/978-3-642-40717-8_28.
(11) Migneault, I.; Dartiguenave, C.; Bertrand, M. J.; Waldron, K. C. Glutaraldehyde: Behavior in Aqueous Solution, Reaction with Proteins, and Application to Enzyme Crosslinking. Biotechniques 2004, 37 (5), 790-802. https://doi.org/10.2144/04375RV01.
(12) Mer, M.; Hodgson, E.; Wallis, L.; Jacobson, B.; Levien, L.; Snyman, J.; Sussman, M. J.; James, M.; van Gelder, A.; Allgaier, R.; et al. Hemoglobin Glutamer-250 (Bovine) in South Africa: Consensus Usage Guidelines from Clinician Experts Who Have Treated Patients. Transfusion. 2016, pp 2631-2636. https://doi.org/10.1111/trf.13726.
(13) Rentko, V. T.; Pearce, L. B.; Moon-Massat, P. F.; Gawryl, M. S. Hemopure® (HBOC-201, Hemoglobin Glutamer-250 (Bovine)): Preclinical Studies. In Blood Substitutes; Elsevier, 2006; pp 424-436. https://doi.org/10.1016/B978-012759760-7/50046-9.
(14) Day, T. K. Current Development and Use of Hemoglobin-Based Oxygen-Carrying (HBOC) Solutions. Journal of Veterinary Emergency and Critical Care. 2003, pp 77-93. https://doi.org/10.1046/j.1435-6935.2003.00084.x.
(15) Napolitano, L. M. Hemoglobin-Based Oxygen Carriers: First, Second or Third Generation? Human or Bovine? Where Are We Now? Hemoglobin-Based Oxygen Carrier HBOC Intensive Care Critical Care Blood Transfusion Hemoglobin. Crit Care Clin 2009, 25, 279-301. https://doi.org/10.1016/j.ccc.2009.01.003.
(16) Marret, E.; Bonnin, P.; Mazoyer, E.; Riou, B.; Jacobs, T.; Coriat, P.; Samama, C. M. The Effects of a Polymerized Bovine-Derived Hemoglobin Solution in a Rabbit Model of Arterial Thrombosis and Bleeding. Anesth. Analg. 2004, 98 (3), 604-610. https://doi.org/10.1213/01.ANE.0000099366.73625.DD.
(17) Driessen, B.; Jahr, J. S.; Lurie, F.; Gunther, R. A. Inadequacy of Low-Volume Resuscitation with Hemoglobin-Based Oxygen Carrier Hemoglobin Glutamer-200 (Bovine) in Canine Hypovolemia. J. Vet. Pharmacol. Ther. 2001, 24 (1), 61-71. https://doi.org/10.1046/j 0.1365-2885.2001.00307.x.
(18) Belcik, B. M.; Palmer, A. F. Vasoconstriction, Hypertension and Oxidative Toxicity Are Regulated by Polymerized Hemoglobin Size. In Hemoglobin-Based Oxygen Carriers as Red Cell Substitutes and Oxygen Therapeutics; 2013; Vol. 9783642407, pp 693-711. https://doi.org/10.1007/978-3-642-40717-8_37.
(19) Jahr, J. S.; Liu, H.; Albert, O. K.; Gull, A.; Moallempour, M., Lim, J.; Gosselin, R. Does HBOC-201 (Hemopure) Affect Platelet Function in Orthopedic Surgery: A Single-Site Analysis from a Multicenter Study. Am. J. Ther. 2010, 17 (2), 140-147. https://doi.org/10.1097/MJT.0b013e3181a2b08d.
(20) Baek, J. H., Zhou, Y.; Harris, D. R., Schaer, D. J.; Palmer, A. F.; Buehler, P. W. Down Selection of Polymerized Bovine Hemoglobins for Use as Oxygen Releasing Therapeutics in a Guinea Pig Model. 2012, 127 (2), 567-581. https://doi.org/10.1093/toxsci/kfs109.
(21) Moore, E. E.; Moore, F. A.; Fabian, T. C.; Bernard, A. C.; Fulda, G. J.; Hoyt, D. B.; Duane, T. M.; Weireter, L J.; Gomez, G. A.; Cipolle, M. D.; et al. Human Polymerized Hemoglobin for the Treatment of Hemorrhagic Shock When Blood Is Unavailable: The USA Multicenter Trial. J. Am. Coll. Surg. 2009, 208 (1), 1-13. https://doi.org/10.1016/j.jamcollsurg.2008.09.023.
(22) Williams, A. T.; Lucas, A.; Muller, C. R.; Munoz, C.; Bolden-Rush, C.; Palmer, A. F.; Cabrales, P. Resuscitation From Hemorrhagic Shock With Fresh and Stored Blood and Polymerized Hemoglobin. Shock 2020, 54 (4), 464-473. https://doi.org/10.1097/SHK.0000000000001530.
(23) Gu, X.; Bolden-Rush, C.; Cuddington, C. T.; Belcher, D. A., Savla, C.; Pires, I. S.; Palmer, A. F. Comprehensive Characterization of Tense and Relaxed Quaternary State Glutaraldehyde Polymerized Bovine Hemoglobin as a Function of Cross-link Density. Biotechnol. Bioeng. 2020, 117(8), 2362-2376. https://doi.org/i0.1002/bit.27382.
(24) Vandegriff, K. D., Malavalli, A.; Wooldridge, J.; Lohman, J.; Winslow, R. M. MP4, a New Nonvasoactive PEG-Hb Conjugate. Transfusion 2003, 43 (4), 509-516. https://doi.org/10.1046/j.1537-2995.2003.00341.x.
(25) CABRALES, P.; FRIEDMAN, J. M. Pegylated Hemoglobins Mechanisms to Avoid Vasoconstriction and Maintain Perfusion. Transfus. Altern. Transfus. Med. 2007, 9 (4), 281-293. https://doi.org/10.1111/j.1778-428X.2007.00090.x.
(26) Savla, C.; Palmer, A. F. Structural Stability and Biophysical Properties of the Mega-Protein Erythrocruorin Are Regulated by Polyethylene Glycol Surface Coverage. Biomacromolecules 2021, 22 (5), 2081-2093. https://doi.org/10.1021/acs.biomac.1c00196.
(27) Vandegriff, K. D., McCarthy, M.; Rohlfs, R. J.; Winslow, R. M. Colloid Osmotic Properties of Modified Hemoglobins: Chemically Cross-Linked versus Polyethylene Glycol Surface-Conjugated. Biophys. Chem. 1997, 69 (1), 23-30. https://doi.org/10.1016/S0301-4622(97)00079-3.
(28) Misra, H.; Lickliter, J.; Kazo, F.; Abuchowski, A. PEGylated Carboxyhemoglobin Bovine (SANGUINATE): Results of a Phase I Clinical Trial. Artif Organs 2014, 38 (8), 702-707. https://doi.org/10.1111/aor.12341.
(29) Björkholm, M.; Fagrell, B.; Przybelski, R.; Winslow, N.; Young, M.; Winslow, R. M. A Phase I Single Blind Clinic Trial of a New Oxygen Transport Agent (MP4), Human Hemoglobin Modified with Maleimide-Activated Polyethylene Glycol. Haematologica. 2005, 90 (4), 505-515.
(30) Caccia, D.; Ronda, L.; Frassi, R.; Perrella, M.; Del Favero, E.; Bruno, S.; Pioselli, B.; Abbruzzetti, S.; Viappiani, C.; Mozzarelli, A. PEGylation Promotes Hemoglobin Tetramer Dissociation. Bioconjug. Chem. 2009, 20 (7), 1356-1366. https://doi.org/10.1021/bc900130f.
(31) Palmer, A. F.; Sun, G.; Harris, D. R. Tangential Flow Filtration of Hemoglobin. Biotechnol. Prog. 2009, 25 (1), 189-199. https://doi.org/10.1002/btpr.119.
(32) Drabkin, D. L.; Austin, J. H. Spectrophotometric Studies Ii. Preparations From Washed Blood Cells; Nitric Oxide Hemoglobin and Sulfhemoglobin. J. Biol. Chem. 1935, 112 (1), 51-65.
(33) Elmer, J.; Harris, D.; Palmer, A. F. Purification of Hemoglobin from Red Blood Cells Using Tangential Flow Filtration and Immobilized Metal Ion Affinity Chromatography. J. Chromatogr. B Anal. Technol. Biomed. Life Sci. 2011, 879 (2), 131-138. https://doi.org/10.1016/j.jchromb.2010.11.021.
(34) Pires, I. S.; Savla, C.; Palmer, A. F. Poly(Ethylene Glycol) Surface-Conjugated Apohemoglobin as a Synthetic Heme Scavenger. Biomacromolecules 2020, 21 (6), 2155-2164. https://doi.org/10.1021/acs.biomac.0c00141.
(35) Savla, C.; Munoz, C.; Hickey, R.; Belicak, M.; Gilbert, C.; Cabrales, P.; Palmer, A. F. Purification of Lumbricus Terrestris Mega-Hemoglobin for Diverse Oxygen Therapeutic Applications. ACS Biomater. Sci. Eng. 2020, 6 (9), 4957-4968. https://doi.org/10.1021/acsbiomaterials.0c01146.
(36) HILL, A. The Possible Effects of the Aggregation of the Molecules of Haemoglobin on Its Dissociation Curves. J Physiol (Lond). Jan. 1, 1910, pp 4-7.
(37) Meng, F.; Kassa, T.; Jana, S.; Wood, F.; Zhang, X.; Jia, Y.; D′Agnillo, F.; Alayash, A. I. Comprehensive Biochemical and Biophysical Characterization of Hemoglobin-Based Oxygen Carrier Therapeutics: All HBOCs Are Not Created Equally. Bioconjug. Chem. 2018, 29 (5), 1560-1575. https://doi.org/10.1021/acs.bioconjchem.8b00093.
(38) Taguchi, K.; Yamasaki, K.; Maruyama, T.; Otagiri, M. Comparison of the Pharmacokinetic Properties of Hemoglobin-Based Oxygen Carriers. J. Funct. Biomater. 2017, 8 (1), 11. https://doi.org/10.3390fjfb8010011.
(39) Umut, E. Surface Modification of Nanoparticles Used in Biomedical Applications. In Modern Surface Engineering Treatments; InTech, 2013. https://doi.org/10.5772/55746.
(40) Wang, Y.; Wang, L.; Yu, W.; Gao, D.; You, G.; Li, P.; Zhang, S.; Zhang, J.; Hu, T.; Zhao, L.; et al. A PEGylated Bovine Hemoglobin as a Potent Hemoglobin-Based Oxygen Carrier. Biotechnol. Prog. 2017, 33 (1), 252-260. https://doi.org/10.1002/btpr.2380.
(41) Buehler, P. W.; Boykins, R. A.; Jia, Y.; Norris, S.; Freedberg, D. I.; Alayash, A. I. Structural and Functional Characterization of Glutaraldehyde-Polymerized Bovine Hemoglobin and Its Isolated Fractions. Anal. Chem. 2005, 77 (11), 3466-3478. https://doi.org/10.1021/ac050064y.
(42) Lou Carmichael, F. J.; Ali, A. C. Y., Campbell, J. A.; Langlois, S. F.; Biro, G. P., Willan, R.; Pierce, C. H., Gerson Greenburg, A. A Phase I Study of Oxidized Raffinose Cross-Linked Human Hemoglobin. Crit. Care Med. 2000, 28 (7), 2283-2292. https://doi.org/10.1097/00003246-200007000-00017.
(43) Napolitano, L. M. Hemoglobin-Based Oxygen Carriers: First, Second or Third Generation? Human or Bovine? Where Are We Now? Critical Care Clinics. 2009, pp 279-301. https://doi.org/10.1016/j.ccc.2009.01.003.
(44) Port6r6, I.; Kocsis, L.; Herman, P.; Caccia, D.; Perrella, M.; Ronda, L.; Bruno, S.; Bettati, S.; Micalella, C.; Mozzarelli, A.; et al. Towards a Novel Haemoglobin-Based Oxygen Carrier: Euro-PEG-Hb, Physico-Chemical Properties, Vasoactivity and Renal Filtration. Biochim. Biophys. Acta-Proteins Proteomics 2008, 1784 (10), 1402-1409. https://doi.org/10.1016/j.bbapap.2008.03.005.
(45) Belcher, D. A.; Cuddington, C. T.; Martindale, E. L.; Pires, I. S.; Palmer, A. F. Controlled Polymerization and Ultrafiltration Increase the Consistency of Polymerized Hemoglobin for Use as an Oxygen Carrier. Bioconjug. Chem. 2020, 31 (3), 605-621. https://doi.org/10.1021/acs.bioconjchem.9b00766.
(46) Tsuruga, M.; Matsuoka, A.; Hachimori, A.; Sugawara, Y.; Shikama, K. The Molecular Mechanism of Autoxidation for Human Oxyhemoglobin. Tilling of the Distal Histidine Causes Nonequivalent Oxidation in the β Chain; 1998; Vol. 273. https://doi.org/10.1074/jbc.273.15.8607.
(47) Alayash, A. I. Mechanisms of Toxicity and Modulation of Hemoglobin-Based Oxygen Carriers. Shock 2019, 52 (1S), 41-49. https:/doi.org/10.1097/SHK.0000000000001044.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A hemoglobin-based oxygen carrier (HBOC) comprising a polymer-functionalized polymerized hemoglobin;
wherein the polymer-functionalized polymerized hemoglobin has a weight average molecular weight of from 500 kDa to 2,000 kDa, as determined by size exclusion (SEC) HPLC.

2. The carrier of claim 1, wherein the polymer-functionalized polymerized hemoglobin is substantially free of low-molecular weight hemoglobin species having a molecular weight of less than 100 kDa.

3. The carrier of claim 2, wherein the polymer-functionalized polymerized hemoglobin is substantially free of low-molecular weight hemoglobin species having a molecular weight of less than 500 kDa.

4. The carrier of claim 1, wherein the polymer-functionalized polymerized hemoglobin exhibits an average hydrodynamic diameter of from 13 nm to 100 nm, as measured by dynamic light scattering, such as from 13 nm to 50 nm, or from 13 nm to 30 nm.

5. The carrier of claim 1, wherein the polymer-functionalized polymerized hemoglobin exhibits a zeta potential of from −20 mV to less than 0 mV, such as from −10 mV to less than 0 mV.

6. The carrier of claim 1, wherein the hemoglobin comprises bovine hemoglobin, procine hemoglobin, human hemoglobin, or recombinant hemoglobin.

7. The carrier of claim 1, wherein the polymerized hemoglobin comprises hemoglobin crosslinked with a dialdehyde, such as glutaraldehyde.

8. The carrier of claim 7, wherein the polymerized hemoglobin is formed by a process that comprises crosslinking hemoglobin with a dialdehyde, such as glutaraldehyde, at a molar ratio of dialdehyde:hemoglobin of from 20:1 to 35:1.

9. The carrier of claim 8, wherein the hemoglobin is substantially in the T-state (tense quaternary state) during the crosslinking.

10. The carrier of claim 8, wherein the hemoglobin is substantially in the R-state (relaxed quaternary state) during the crosslinking.

11. The carrier of claim 1, wherein the polymerized hemoglobin further comprises one or more antioxidant proteins co-polymerized with the hemoglobin, such as a peroxiredoxin, a superoxide dismutase, a catalase, or a combination thereof.

12. The carrier of claim 1, wherein the polymer-functionalized polymerized hemoglobin comprises a polymer or oligomer covalently conjugated to the polymerized hemoglobin.

13. The carrier of claim 12, wherein the polymer or oligomer comprises a polyalkylene oxide, such as a polyethylene glycol (PEG).

14. The carrier of claim 12, wherein the polymer or oligomer comprises a zwitterionic polymer, such as a polycarboxybetaine (PCB) or a polysulfobetaine (PSB).

15. The carrier of claim 12, wherein the polymer or oligomer comprises a carbohydrate such as a dextran.

16. The carrier of claim 1, wherein the polymer-functionalized polymerized hemoglobin comprises a polyalkylene oxide (PAO)-functionalized polymerized hemoglobin.

17. The carrier of claim 16, wherein the PAO-functionalized polymerized hemoglobin comprises a polyalkene oxide covalently conjugated to the polymerized hemoglobin.

18. The carrier of claim 16, wherein the PAO is polyethylene glycol (PEG) according to the formula of H(OCH₂CH₂)ₙOH, where n is greater than or equal to 4.

19. The carrier of claim 18, wherein n is from 10 to 250, such as from 75 to 125.

20. The carrier of claim 16, wherein the PAO-functionalized polymerized hemoglobin comprises polymerized hemoglobin conjugated with malemidyl-activated polyethylene glycol (Mal-PEG), and is defined by Formula I below
PolyHb-(S—Y—R—CH₂—CH₂—[O—CH₂—CH₂]ₙ—O—CH₃)ₘ Formula I
wherein
PolyHb represent polymerized hemoglobin;
S represents a surface thiol group;
Y represents a covalent bond between the polymerized hemoglobin and PEG;
R is a linker;
n is an integer of from 10 to 250, such as from 75 to 125; and
m is an integer of greater than 2.

21. The carrier of claim 20, wherein m is from 4 to 5.

22. The carrier of claim 1, wherein the polymer-functionalized polymerized hemoglobin was polymerized in the T-state (tense quaternary state).

23. The carrier of claim 22, wherein the polymer-functionalized polymerized hemoglobin exhibits a P₅₀ of from 15 mm Hg to 40 mm Hg.

24. The carrier of claim 22, wherein the polymer-functionalized polymerized hemoglobin exhibits a k_{off,O2} of from 10 s⁻¹ to 40 s⁻¹.

25. The carrier of claim 1, wherein the polymer-functionalized polymerized hemoglobin was polymerized in the R-state (relaxed quaternary state).

26. The carrier of claim 25, wherein the polymer-functionalized polymerized hemoglobin exhibits a P₅₀ of 1.0±0.5 mm Hg.

27. The carrier of claim 25, wherein the polyalkylene oxide (PAO)-functionalized polymerized hemoglobin exhibits a k_{off,O2} of from 7 s⁻¹ to 20 s⁻¹.

28. A method of producing a hemoglobin-based oxygen carrier (HBOC), the method comprising:
(i) contacting hemoglobin with a multifunctional cross-linking agent to form a solution comprising polymerized hemoglobin;
(ii) filtering the solution comprising polymerized hemoglobin by ultrafiltration against a first filtration membrane having a pore size that separates the polymerized hemoglobin from low-molecular weight hemoglobin species, reactants, and reaction byproducts, thereby forming a retentate fraction comprising the polymerized hemoglobin and a permeate fraction comprising impurities;
(iii) covalently conjugating one or more polymers to the polymerized hemoglobin to form a solution comprising a polymer-functionalized polymerized hemoglobin; and
(iv) filtering the solution comprising the polymer-functionalized polymerized hemoglobin by ultrafiltration against a second filtration membrane having a pore size that separates the polymer-functionalized polymerized hemoglobin from low-molecular weight impurities, reactants, and reaction byproducts, thereby forming a retentate fraction comprising the polymer-functionalized polymerized hemoglobin and a permeate fraction comprising impurities.

29-49. (canceled)

50. A hemoglobin-based oxygen carrier (HBOC) prepared by the method of claim 28.

51. A pharmaceutical composition comprising a hemoglobin-based oxygen carrier (HBOC) defined by claim 1.

52. (canceled)

53. (canceled)

54. (canceled)

55. A method of treating a subject comprising administering to the subject a carrier defined by claim 1.

56. (canceled)

57. A method of preserving a biological tissue sample ex vivo, the method comprising:
contacting the tissue sample ex vivo with a carrier defined by claim 1.

58. (canceled)

59. (canceled)

60. (canceled)
